(19) United States
(12) Patent Application Publication
(10) Pub. No.: US 2024/0390131 A1
(43) Pub. Date: Nov. 28, 2024

(54) VESSEL ACCESS CATHETER

(71) Applicant: WALZMAN INNOVATIONS, LLC, Boynton Beach, FL (US)

(72) Inventor: Daniel Ezra Walzman, Teaneck, NJ (US)

(73) Assignee: WALZMAN INNOVATIONS, LLC, Boynton Beach, FL (US)

(21) Appl. No.: 18/794,013

(22) Filed: Aug. 5, 2024

Related U.S. Application Data
(62) Division of application No. 16/828,312, filed on Mar. 24, 2020.
Application No. 16/828,312 is a continuation-in-part of application No. 15/732,130, filed on Sep. 20, 2017, now abandoned.
Application No. 15/732,130 is a continuation of application No. 15/167,304, filed on May 27, 2016, now abandoned.
Application No. 15/167,304 is a continuation of application No. 15/158,341, filed on May 18, 2016, now abandoned.

(51) Int. Cl. A61F 2/07; A61F 2/06; A61F 2/82; A61F 2/95; A61F 2/958

(52) U.S. Cl. CPC A61F 2/07; A61F 2/06; A61F 2/82; A61F 2/95; A61F 2002/061; A61F 2/958; A61F 2210/0014; A61F 2230/0013; A61F 2230/006; A61F 2250/0039

(57) ABSTRACT

The described invention provides an endovascular device comprising a tube comprising at least one side-hole, a first segment comprising a primary opening and a second segment. The side-hole and the first segment form a working lumen. The second segment forms a support lumen where the support lumen is effective: (i) to provide stability to the working lumen of the endovascular device; (ii) to anchor the endovascular device within a blood vessel; (iii) to prevent catheter coil prolapse due to a counterforce against the endovascular device by one or more additional endovascular devices being delivered through the endovascular device to a more distal location or by a prolapsed coil tail of the endovascular device; and (iv) to facilitate placement of the second endovascular device distally.

### CROSS REFERENCE TO RELATED APPLICATIONS

This application is a continuation-in-part of U.S. application Ser. No. 15/732,130, filed Sep. 20, 2017, which is a continuation of U.S. application Ser. No. 15/167,304, filed May 27, 2016, which claims the benefit of priority to U.S. application Ser. No. 15/158,341, filed May 18, 2016, the contents of each of which are incorporated by reference herein in its entirety.

### FIELD OF THE INVENTION

The described invention relates generally to endovascular devices and endovascular delivery systems, as well as methods of using them.

### BACKGROUND OF THE INVENTION

### Blood Vessel Structure and Function

Blood vessels are dynamic structures that constrict, relax, pulsate, and proliferate. Within the body, blood vessels form a closed delivery system that begins and ends at the heart. There are three major types of blood vessels: (i) arteries; (ii) capillaries and (iii) veins. As the heart contracts, it forces blood into the large arteries leaving the ventricles. Blood then moves into smaller arteries successively, until finally reaching the smallest branches, the arterioles, which feed into the capillary beds of organs and tissues. Blood drains from the capillaries into venules, the smallest veins, and then into larger veins that merge and ultimately empty into the heart.

Arteries carry blood away from the heart and “branch” as they form smaller and smaller divisions. In contrast, veins carry blood toward the heart and “merge” into larger and larger vessels approaching the heart. In the systemic circulation, arteries carry oxygenated blood and veins carry oxygen-poor blood. In the pulmonary circulation, the opposite is true. The arteries (still defined as the vessels leading away from the heart), carry oxygen-poor blood to the lungs, and the veins carry oxygen-rich blood from the lungs to the heart.

The only blood vessels that have intimate contact with tissue cells in the human body are capillaries. In this way, capillaries help serve cellular needs. Exchanges between the blood and tissue cells occur primarily through the thin capillary walls.

The walls of most blood vessels (the exception being the smallest vessels, e.g., venules), have three layers, or tunics, that surround a central blood-containing space called the vessel lumen.

The innermost tunic (layer) is the tunica intima. The tunica intima contains the endothelium, the simple squamous epithelium that lines the lumen of all vessels. The endothelium is continuous with the endocardial lining of the heart, and its flat cells fit closely together, forming a slippery surface that minimizes friction so blood moves smoothly through the lumen. In vessels larger than 1 mm in diameter, a sub-endothelial layer, consisting of a basement membrane and loose connective tissue, supports the endothelium.

The middle tunic (layer), the tunica media, is mostly circularly arranged smooth muscle cells and sheets of elastin. The activity of the smooth muscle is regulated by sympathetic vasomotor nerve fibers of the autonomic nervous system. Depending on the body's needs at any given time, regulation causes either vasoconstriction (lumen diameter decreases) or vasodilation (lumen diameter increases). The activities of the tunica media are critical in regulating the circulatory system because small changes in vessel diameter greatly influence blood flow and blood pressure. Generally, the tunica media is the bulkiest layer in arteries, which bear the chief responsibility for maintaining blood pressure and proper circulation.

The outer layer of a blood vessel wall, the tunica externa, is primarily composed of collagen fibers that protect the vessel, reinforce the vessel, and anchor the vessel to surrounding structures. The tunica externa contains nerve fibers, lymphatic vessels, and elastic fibers (e.g., in large veins). In large vessels, the tunica externa contains a structure known as the vasa vasorum, which literally means “vessels of vessels”. The vasa vasorum nourishes external tissues of the blood vessel wall. Interior layers of blood vessels receive nutrients directly from blood in the lumen (See, e.g., The Cardiovascular System at a Glance, 4^{th} Edition, Philip I. Aaronson, Jeremy P. T. Ward, Michelle J. Connolly, November 2012, 2012, Wiley-Blackwell, Hoboken, N.J.).

### Cerebral Arteries

FIGS. 8 and 9 show schematic illustrations of the brain's blood vessels. Each cerebral hemisphere is supplied by an internal carotid artery, which arises from a common carotid artery beneath the angle of the jaw, enters the cranium through the carotid foramen, traverses the cavernous sinus penetrates the dura and divides into the anterior and middle cerebral arteries. The large surface branches of the anterior cerebral artery supply the cortex and white matter of the inferior frontal lobe, the medial surface of the frontal and parietal lobes and the anterior corpus callosum. Smaller penetrating branches supply the deeper cerebrum and diencephalon, including limbic structures, the head of the caudate, and the anterior limb of the internal capsule. The large surface branches of the middle cerebral artery supply most of the cortex and white matter of the hemisphere's convexity, including the frontal, parietal, temporal and occipital lobes, and the insula. Smaller penetrating branches supply the deep white matter and diencephalic structures such as the posterior limb of the internal capsule, the putamen, the outer globus pallidus, and the body of the caudate. After the internal carotid artery emerges from the cavernous sinus, it also gives off the anterior choroidal artery, which supplies the anterior hippocampus and, at a caudal level, the posterior limb of the internal capsule. Each vertebral artery arises from a subclavian artery, enters the cranium through the foramen magnum, and gives off an anterior spinal artery and a posterior inferior cerebellar artery. The vertebral arteries join at the junction of the pons and the medulla to form the basilar artery, which at the level of the pons gives off the anterior inferior cerebellar artery and the internal auditory artery, and, at the midbrain, the superior cerebellar artery. The basilar artery then divides into the two posterior cerebral arteries. The large surface branches of the posterior cerebral arteries supply the inferior temporal and medial occipital lobes and the posterior corpus callosum; the smaller penetrating branches of these arteries supply diencephalic structures, including the thalamus and the subthalamic nuclei, as well as part of the midbrain (see Principles of Neural Sciences, 2d Ed., Eric R. Kandel and James H. Schwartz, Elsevier Science Publishing Co., Inc., New York, pp. 854-56 (1985)).

Interconnections between blood vessels (anastomoses) protect the brain when part of its vascular supply is compromised. At the circle of Willis, the two anterior cerebral arteries are connected by the anterior communicating artery and the posterior cerebral arteries are connected to the internal carotid arteries by the posterior communicating arteries. Other important anastomoses include connections between the ophthalmic artery and branches of the external carotid artery through the orbit, and connections at the brain surface between branches of the middle, anterior, and posterior cerebral arteries (Principles of Neural Sciences, 2d Ed., Eric R. Kandel and James H. Schwartz, Elsevier Science Publishing Co., Inc., New York, pp. 85456 (1985)).

### Femoral Artery

The femoral artery is the main artery that provides oxygenated blood to the tissues of the leg. It passes through the deep tissues of the femoral (or thigh) region of the leg parallel to the femur.

The common femoral artery is the largest artery found in the femoral (thigh) region of the body. It begins as a continuation of the external iliac artery at the inguinal ligament which serves as the dividing line between the pelvis and the leg. From the inguinal ligament, the femoral artery follows the medial side of the head and neck of the femur inferiorly and laterally before splitting into the deep femoral artery and the superficial femoral artery.

The superficial femoral artery flexes to follow the femur inferiorly and medially. At its distal end, it flexes again and descends posterior to the femur before forming the popliteal artery of the posterior knee and continuing on into the lower leg and foot. Several smaller arteries branch off from the superficial femoral artery to provide blood to the skin and superficial muscles of the thigh.

The deep femoral artery follows the same path as the superficial branch, but follows a deeper path through the tissues of the thigh, closer to the femur. It branches off into the lateral and medial circumflex arteries and the perforating arteries that wrap around the femur and deliver blood to the femur and deep muscles of the thigh. Unlike the superficial femoral artery, none of the branches of the deep femoral artery continue into the lower leg or foot.

Like most blood vessels, the femoral artery is made of several distinct tissue layers that help it to deliver blood to the tissues of the leg. The innermost layer, known as the endothelium or tunica intima, is made of thin, simple squamous epithelium that holds the blood inside the hollow lumen of the blood vessel and prevents platelets from sticking to the surface and forming blood dots. Surrounding the tunica intima is a thicker middle layer of connective tissues known as the tunica media. The tunica media contains many elastic and collagen fibers that give the femoral artery its strength and elasticity to withstand the force of blood pressure inside the vessel Visceral muscle in the tunica media may contract or relax to help regulate the amount of blood flow. Finally, the tunica externa is the outermost layer of the femoral artery that contains many collagen fibers to reinforce the artery and anchor it to the surrounding tissues so that it remains stationary.

The femoral artery is classified as an elastic artery, meaning that it contains many elastic fibers that allow it to stretch in response to blood pressure. Every contraction of the heart causes a sudden increase in the blood pressure in the femoral artery, and the artery wall expands to accommodate the blood. This property allows the femoral artery to be used to detect a person's pulse through the skin (See, e.g., The Cardiovascular System at a Glance, 4^{th} Edition, Philip I. Aaronson, Jeremy P. T. Ward, Michelle J. Connolly, November 2012, © 2012, Wiley-Blackwell, Hoboken, N.J.).

### Use of the Femoral Artery for Endovascular Procedures

Endovascular diagnostic and therapeutic procedures are generally performed through the femoral artery. Some of the reasons for this generalized approach include its location, easy approach for puncture and hemostasis, low rate of complications, technical case, wide applicability and relative patient comfort (Alvarez-Tostado J. A. et al. Journal of Vascular Surgery 2009; 49 (2): 378-385). Femoral puncture also allows access to virtually all of the arterial territories and affords favorable ergonomics for the operator in most instances (Alvarez-Tostado J. A. et al. Journal of Vascular Surgery 2009; 49 (2): 378-385).

### Brachial Artery

The brachial artery is a major blood vessel located in the upper arm and is the main supplier of blood to the arm and hand. It continues from the axillary artery at the shoulder and travels down the underside of the arm. Along with the medial cubital vein and bicep tendon, it forms the cubital fossa, a triangular pit on the inside of the elbow. Below the cubital fossa, the brachial artery divides into two arteries running down the forearm: the ulnar and the radial; the two main branches of the brachial artery. Other branches of the brachial artery include the inferior ulnar collateral, profunda brachii, and superior ulnar arteries (See, e.g., The Cardiovascular System at a Glance, 4^{th} Edition, Philip I. Aaronson, Jeremy P. T. Ward, Michelle J. Connolly, November 2012, © 2012, Wiley-Blackwell, Hoboken, N.J.).

### Use of the Brachial Artery for Endovascular Procedures

Brachial artery access is a critical component of complex endovascular procedures, especially in instances where femoral access is difficult or contraindicated, such as the absence of palpable femoral pulses, severe common femoral occlusive disease, recent femoral intervention or surgery or femoral aneurysms/pseudoaneurysms. It is a straightforward procedure with a high success rate for percutaneous cannulation (Alvarez-Tostado J. A. et al. Journal of Vascular Surgery 2009; 49 (2): 378-385). However, there is a general reluctance to puncture the right brachial artery due to the need to navigate through the innominate artery and arch and due to the risk for complications such as direct nerve trauma and ischemic occlusion resulting in long-term disability (Alvarez-Tostado J. A. et al. Journal of Vascular Surgery 2009; 49 (2): 378-385; Cousins T. R. and O'Donnell J. M. AANA Journal 2004; 72 (4): 267-271). To minimize ischemic injury to the distal arm, the radial artery has become a more common access site for percutaneous vascular procedures, as the distal radial artery territory has robust collateral circulation.

### Endovascular Intervention

The current standard for therapeutic recanalization and reperfusion in vascular disease and acute stroke is to perform endovascular interventions via a transfemoral approach, meaning, starting a catheter in the femoral artery at the groin, proceeding through the aorta and carotid artery to the affected blood vessel. All existing devices are designed to be used from this starting point and surgeons are most familiar and comfortable with this route.

### Mechanical Endovascular Intervention in Coronary Artery Disease (CAD)

### Percutaneous Coronary Intervention (PCI)

Percutaneous coronary intervention (PCI) is a nonsurgical method for coronary artery revascularization. PCI methods include balloon angioplasty, coronary stenting, atherectomy (devices that ablate plaque), thrombectomy (devices that remove clots from blood vessels) and embolic protection (devices that capture and remove embolic debris).

### Balloon Angioplasty

Balloon angioplasty involves advancing a balloon-tipped catheter to an area of coronary narrowing, inflating the balloon, and then removing the catheter after deflation. Balloon angioplasty can reduce the severity of coronary stenosis, improve coronary flow, and diminish or eliminate objective and subjective manifestations of ischemia (Losordo D. W. et al. Circulation 1992 December 86 (6): 1845-58). The mechanism of balloon angioplasty action involves three events: plaque fracture, compression of the plaque, and stretching of the vessel wall. These lead to expansion of the external elastic lumina and axial plaque redistribution along the length of the vessel (Losordo D. W. et al. Circulation 1992 December 86 (6): 1845-58).

### Coronary Stenting

Coronary stents are metallic scaffolds that are deployed within a diseased coronary artery segment to maintain wide luminal patency. They were devised as permanent endoluminal prostheses that could seal dissections, create a predictably large initial lumen, and prevent early recoil and late vascular remodeling (Krajcer Z. and Howell M. H. Tex Heart Inst J. 2000; 27 (4): 369-385).

Drug-eluting stents (DES s) elute medication to reduce restenosis (the recurrence of abnormal narrowing of a blood vessel) within the stents. Local release of rapamycin and its derivatives or of paclitaxel from a polymer matrix on the stent during the 30 days after implantation has been shown to reduce inflammation and smooth muscle cell proliferation within the stent, decreasing in-stent late loss of luminal diameter from the usual 1 mm to as little as 0.2 mm (Stone G. W. et al. N Engl J Med. 2007 Mar. 8. 356 (10): 998-1008). This dramatically lowers the restenosis rate after initial stent implantation or after secondary implantation of a DES for an in-stent restenosis (Stone G. W. et al. N Engl J Med. 2007 Mar. 8. 356 (10): 998-1008).

Coronary stents are used in about 90% of interventional procedures. Stent-assisted coronary intervention has replaced coronary artery bypass graft (CABG) as the most common revascularization procedure in patients with coronary artery disease (CAD) and is used in patients with multi-vessel disease and complex coronary anatomy (Kalyanasundaram A. et al. Medscape Dec. 16, 2014; article 164682; emedicine.medscape.com/article/164682-overview #a3).

### Atherectomy 10

The directional coronary atherectomy (DCA) catheter was first used in human peripheral vessels in 1985 and in coronary arteries in 1986. In this procedure, a low-pressure positioning balloon presses a windowed steel housing against a lesion; any plaque that protrudes into the window is shaved from the lesion by a spinning cup-shaped cutter and trapped in the device's nose cone (Hinohara T. et al. Circulation 1990 March 81 (3 Suppl): IV79-91).

Rotational atherectomy uses a high-speed mechanical rotational stainless-steel burr with a diamond chip—embedded surface. The burr is attached to a hollow flexible drive shaft that permits it to be advanced over a steerable guide wire with a platinum coil tip. The drive shaft is encased within a TEFLON® sheath through which a flush solution is pumped to lubricate and cool the drive shaft and burr. A compressed air turbine rotates the drive shaft at 140,000-200,000 rpm during advancement across a lesion (Hinohara T. et al. Circulation 1990 March 81 (3 Suppl): IV79-91)

### Laser Ablation

In laser ablation, an intense light beam travels via optical fibers within a catheter and enters the coronary lumen. After the target lesion is crossed with the guide wire, the laser catheter is advanced to the proximal end of the lesion. Blood and contrast medium are removed from the target vessel by flushing with saline before activating the laser (Kalyanasundaram A. et al. Medscape Dec. 16, 2014; article 164682; emedicine.medscape.com/article/164682-overview #a3).

### Mechanical Thrombectomy

Intracoronary thrombi may be treated with mechanical thrombectomy devices. These include rheolytic, suction and ultrasonic thrombectomy devices.

In rheolytic thrombectomy, high-speed water jets create suction via the Bernoulli-Venturi effect. The jets exit orifices near the catheter tip and spray back into the mouth of the catheter, creating a low-pressure region and intense suction. This suction pulls surrounding blood, thrombus, and saline into the tip opening and propels particles proximally through the catheter lumen and out of the body (Kalyanasundaram A. et al. Medscape Dec. 16, 2014; article 164682; emedicine.medscape.com/article/164682-overview #a3).

The catheters used for suction thrombectomy act via manual aspiration. These catheters are advanced over a wire to the intracoronary thrombus then passed through the thrombus while suction is applied to a hole in the catheter tip. Large intact thrombus fragments can be removed by means of this technique (Kalyanasundaram A. et al. Medscape Dec. 16, 2014; article 164682; emedicine.medscape.com/article/164682-overview #a3).

Ultrasonic thrombectomy involves the use of ultrasonic vibration to induce cavitation that can fragment a thrombus into smaller components (Choi S. W. et al. J. Intery Cardiol. 2006 Feb. 19 (1): 87-92).

### Embolization Protection

Embolization (the passage of an embolus (blood clot) within the blood stream) can be caused by the manipulation of guidewires, balloons, and stents across complex atherosclerotic carotid artery lesions (Krajcer Z. and Howell M. H. Tex Heart Inst J. 2000; 27 (4): 369-385). Several devices have been developed to trap such embolic material and remove it from the circulation.

The PercuSurge Guardwire is a device that consists of a 0.014- or 0.018-inch angioplasty guidewire constructed of a hollow nitinol hypotube. Incorporated into the distal wire segment is an inflatable balloon capable of occluding vessel flow. The proximal end of the wire incorporates a Microseal™ that allows inflation and deflation of the distal occlusion balloon. When the Microseal adapter is detached, the occlusion balloon remains inflated, at which time angioplasty and stenting are performed. An aspiration catheter can be advanced over the wire into the vessel, and manual suction is applied to retrieve particulate debris (Krajcer Z. and Howell M. H. Tex Heart Inst J. 2000; 27 (4): 369-385).

The Medicorp device consists of a protection balloon and a dilation balloon that can be used over a 0.014-inch coronary guidewire. Occlusion above the lesion and below the lesion creates a dilation zone without a flow, which is aspirated and cleared of atherosclerotic debris (Krajcer Z. and Howell M. H. Tex Heart Inst J. 2000; 27 (4): 369-385).

### Endovascular Treatment of Abdominal Aortic Aneurysms (AAA)

Two endoluminal AAA exclusion stent graft systems have received FDA approval: (i) the Ancure™ Endograft System (Guidant/EVT; Menlo Park, Calif.); and (ii) the AneuRx™ device (Medtronic AVE; Santa Rosa, Calif.) (Krajcer Z. and Howell M. H. Tex Heart Inst J. 2000; 27 (4): 369-385). Both are over-the-wire systems that require bilateral femoral artery access.

The Ancure™ stent graft is an unsupported, single piece of woven DACRON® fabric. The graft is bifurcated and has no intra-graft junctions. The main device is delivered through a 24-Fr introducer sheath; a 12-Fr sheath is required to facilitate the deployment of the contralateral iliac limb. The graft is attached via a series of hooks that are located at the proximal aortic end and at both iliac ends. The hooks are seated transmurally (passing through the vessel wall) in the aorta and the iliac arteries, initially by minimal radial force, and then affixed by low-pressure balloon dilation. Radiopaque markers are located on the body of the graft for correct alignment and positioning (Krajcer Z. and Howell M. H. Tex Heart Inst J. 2000; 27 (4): 369-385).

The AncuRx™ device is a modular 2-piece system composed of a main bifurcation segment and a contralateral iliac limb. The graft is made of thin-walled woven polyester that is fully supported by a self-expanding nitinol exoskeleton. Attachment is accomplished by radial force at the attachment sites, which causes a frictional seal. The main bifurcated body is delivered through a 21-Fr sheath, and the contralateral limb requires a 16-Fr sheath. The body of the graft has radiopaque markers that facilitate correct alignment and positioning (Krajcer Z. and Howell M. H. Tex Heart Inst J. 2000; 27 (4): 369-385).

### Mechanical Endovascular Neurointervention

### Mechanical Thrombectomy

Mechanical thrombectomy (excision of a dot from a blood vessel) devices remove occluding thrombi (blood dots) from the target vessel by a catheter. Subgroups include (1) suction thrombectomy devices that remove occlusions from the cerebral vessels by aspiration (Proximal Thrombectomy) and (2) dot removal devices that physically seize cerebral thrombi and drag them out of the cerebral vessels (Distal Thrombectomy) (Gralla J. et al. Stroke 2006; 37:3019-24; Brekenfeld C. et al. Stroke 2008; 39:1213-9).

### Proximal Endovascular Thrombectomy

Manual suction thrombectomy is performed by moving forward an aspiration catheter at the proximal surface of the thrombus (Singh P. et al. J Neurosci Rural Pract. 2013 July-September; 4 (3): 298-303). Manual aspiration is then carried out and the aspiration catheter is taken back under continuous negative pressure. The Penumbra System (Penumbra, Almeda, Calif. USA) is a variation of the manual proximal aspiration method which comprises a dedicated reperfusion catheter attached to a pumping system applying constant aspiration. A second retriever device is similar to a stent and is utilized to take out the resistant dot (Singh P. et al. J Neurosci Rural Pract 2013 July-September; 4 (3): 298-303). The time window for neuroradiological intervention is 8 hours after stroke onset in patients not eligible for intravenous thrombolysis or in patients where intravenous thrombolysis was unsuccessful (Singh P. et al. J Neurosci Rural Pract 2013 July-September; 4 (3): 298-303).

The Penumbra System™ has been examined in a number of clinical trials. The Penumbra Pivotal Stroke Trial was a prospective, single-arm, multicenter study that recruited 125 stroke patients (mean NIHSS 18) within 8 hours of symptom onset and was successful in 81.6% of treated vessels (Penumbra Pivotal Stroke Trial Investigators: The Penumbra pivotal stroke trial: Safety and effectiveness of a new generation of mechanical devices for clot removal in intracranial large vessel occlusive disease. Stroke 2009; 40:2761-8). However, a good clinical outcome at 90 days was attained in only 25% of patients and in 29% of patients with successful recanalization (the process of restoring flow to or reuniting an interrupted channel such as a blood vessel) of the target vessel (Penumbra Pivotal Stroke Trial Investigators: The penumbra pivotal stroke trial: Safety and effectiveness of a new generation of mechanical devices for dot removal in intracranial large vessel occlusive disease. Stroke. 2009; 40:2761-8). Poor clinical results occurred despite comparatively better recanalization rates as evidenced by a mortality rate of 32.8% and the occurrence of symptomatic intracerebral hemorrhage (ICH) in 11.2% (Penumbra Pivotal Stroke Trial Investigators: The penumbra pivotal stroke trial: Safety and effectiveness of a new generation of mechanical devices for dot removal in intracranial large vessel occlusive disease. Stroke. 2009; 40:2761-8).

### Distal Endovascular Thrombectomy

Distal thrombectomy is a technically difficult procedure (Singh P. et al. J Neurosci Rural Pract. 2013 July-September; 4 (3): 298-303). A number of clinical studies have been carried out using the MERCI (Mechanical Embolus Removal in Cerebral Ischemia) RETRIEVER® device (Concentric Medical, Mountain View, USA), which was the earliest distal thrombectomy device approved by the United States Food and Drug Administration (FDA) (Singh P. et al. J Neurosci Rural Pract. 2013 July-September; 4 (3): 298-303). In the initial stage of the procedure, the occlusion site must be traversed with a microcatheter so as to deploy the device beyond the thrombus. The MERCI RETRIEVER® device is pulled back into the thrombus and positioned within the clot. Next, the MERCI RETRIEVER® and the trapped clot are withdrawn, initially into the positioning catheter and then out of the patient's body (Singh P. et al. J Neurosci Rural Pract. 2013 July-September; 4 (3): 298-303). Proximal balloon occlusion by means of a balloon guide catheter and aspiration during retrieval of the Merci device is done for the majority of cases in order to prevent thromboembolic complications (Nogueira R. G. et al. Am J Neuroradiol. 2009; 30:649-61; Nogueira R. G. et al. Am J Neuroradiol. 2009; 30:859-7). During in vivo experimental studies, the distal technique was shown to be more efficient as compared to proximal manual aspiration (Gralla J. et al. Stroke 2006; 37:3019-24).

The MERCI RETRIEVER® clinical trial was a 25-site, uncontrolled, technical efficacy trial (Smith W. S. et al. Stroke 2005; 36:1432-8). The trial incorporated 151 patients with occlusion of the internal carotid artery or vertebral and basilar arteries, who did not qualify for intra-arterial therapy (IAT) within 8 hours of symptom onset (Smith W. S. et al. Stroke 2005; 36:1432-8). Successful recanalization was accomplished in 46%, with excellent clinical outcome in 27.7% of patients (Smith W. S. et al. Stroke 2005; 36:1432-8). Successful recanalization was linked with distinctly better clinical outcomes. Average procedure time was 2.1 hours, with clinically noteworthy procedural complications occurring in 7.1% and a rate of symptomatic intracranial hemorrhage (ICH) occurring in 7.8% of patients (Smith W. S. et al. Stroke 2005; 36:1432-8). Despite good clinical outcome, limitations of this device include operator learning curve, the need to traverse the occluded artery to deploy the device distal to the occlusion, the duration required to perform multiple passes with device, clot fragmentation and passage of an embolus within the bloodstream (Meyers P. M. et al. Circulation 2011; 123:2591-2601).

### Self-Expanding Stents

Until recently, intracranial stenting was restricted to off-label use of balloon-mounted stents intended for cardiac circulation (Singh P. et al. J Neurosci Rural Pract. 2013 July-September; 4 (3): 298-303). These stents are not ideal for treating intracranial disease due to their rigidity which makes navigation in the convoluted intracranial vessels difficult (Singh P. et al. J Neurosci Rural Pract. 2013 July-September; 4 (3): 298-303). Self-expanding intracranial stents permit stenting in acute stroke that is unmanageable with conventional treatment regimens. The clot occluding the vessel is outwardly displaced by the side of the vessel wall and becomes trapped in the interstices of a self-expanding stent (SES). Wingspan™ (Stryker), Neuroform® (Stryker, Kalamazoo, Mich.), and Cordis Enterprise™ (Cordis Neurovascular, Fremont, Calif.) self-expanding stenting systems have improved steering, cause a reduced amount of vasospasm, and cause a reduced amount of side-branch occlusions as compared to balloon-inflated stents Singh P. et al. J Neurosci Rural Pract. 2013 July-September; 4 (3): 298-303). Drawbacks of this method include delayed in-stent thrombosis, the use of platelet inhibitors which may cause intracerebral hemorrhage (ICH) and perforator occlusion from relocation of the thrombus after stent placement (Samaniego E. A. et al. Front Neurol. 2011; 2:1-7; Fitzsimmons B. F. et al. Am J Neuroradiol. 2006; 27:1132-4; Levy E. I. et al. Neurosurgery 2006; 58:458-63; Zaidat O. O. et al. Stroke 2008; 39:2392-5).

### Retrievable Thrombectomy Stents

Retrievable thrombectomy stents are self-expandable, re-sheathable, and re-constrainable stent-like thrombectomy devices which combine the advantages of intracranial stent deployment with immediate reperfusion and subsequent retrieval with definitive clot removal from the occluded artery (Singh P. et al. J Neurosci Rural Pract. 2013 July-September; 4 (3): 298-303). Removal of the device circumvents the drawbacks associated with permanent stent implantation. These include the requirement for double anti-platelet medication, which potentially adds to the risk of hemorrhagic complications and the risk of in-stent thrombosis or stenosis. The application of retrievable thrombectomy stents is analogous to that of intracranial stents. Under general anesthesia, using a transfemoral approach, a guide catheter is positioned in the proximal internal carotid artery. A guide wire is advanced coaxially over a microcatheter within the blocked intracranial vessel and navigated past the thrombus. The microcatheter is then advanced over the wire through the clot, and the guide wire is substituted for the embolectomy device Singh P. et al., J Neurosci Rural Pract. 2013 July-September; 4 (3): 298-303). The revascularization device is placed with the middle third of the device residing within the thrombus formation. The radial force of the stent retriever is able to create a channel by squeezing the thrombus and is able to partially restore blood flow to the distal territory in the majority of cases, producing a channel for a temporary bypass (Singh P. et al., J Neurosci Rural Pract. 2013 July-September; 4 (3): 298-303). The device is usually left in place for an embedding time of up to 10 minutes, permitting entrapment of the thrombus within the stent struts. To extract the thrombus, the unfolded stent and the microcatheter are slowly dragged into the guide catheter with flow reversal by continuous aspiration with a 50-mil syringe from the guide catheter Singh P. et al. J Neurosci Rural Pract. 2013 July-September; 4 (3): 298-303). The designs of these stents differ in terms of radial strength, design of the proximal and distal stent aperture, stent cell design, material and supplementary intraluminal struts (Mordasini P. et al. Am J Neuroradiol 2011; 32:294-300; Brekenfeld C. et al. Am J Neuroradiol. 201; 2:1269-73; Mordasini P. et al. Am J Neuroradiol. 2013; 34:153-8). Despite the potential to diminish procedure time and to improve recanalization rates, drawbacks to using these devices remain. For example, the TREVO 2 study (Thrombectomy Revascularisation of Large Vessel Occlusions in AIS) was an open label, multi-center trial evaluating the efficacy of the Trevo Pro retriever (Stryker Neurovascular, Fremont, USA) with the Merci device in patients with large vessel ischemic stroke (Nogueira R. G. et al. Lancet 2012; 380:1231-40). Symptomatic ICH occurred in 6.8% in the Trevo group and in 8.9% of the Merci group, with mortality rates of 33% an 24% respectively. The outcome of this trial sustains the supposition that there are unique mechanical mechanisms of action and consequently dissimilar success and efficacy rates depending on the thrombectomy approaches applied (Singh P. et al. J Neurosci Rural Pract. 2013 July-September; 4 (3): 298-303).

Although mechanical endovascular neurointerventions using a transfemoral approach are the current standard for the treatment of acute stroke, it is difficult to access the left internal carotid artery via these transfemoral techniques when an aortic arch variation occurs. A similar transfemoral access problem can occur when vertebral arteries arise at an acute angle from the subclavian artery.

### Aortic Arch

### Normal Anatomy

The most common aortic arch branching pattern in humans consists of three great vessels originating from the arch of the aorta. The first branch is the innominate artery (brachiocephalic artery), which branches into the right subclavian artery and the right common carotid artery. The second branch in the most common pattern is the left common carotid artery, and the last branch is the left subclavian artery (Layton K. F. Am J Neuroradiol. 2006; 27:1541-1542) (FIG. 3).

### Variant Anatomy of the Aortic Arch

### Hypoplastic Ascending Aorta

Hypoplasia (underdevelopment or incomplete development) of the ascending aorta usually occurs concomitant with hypoplastic left heart syndrome (HLHS). HLHS comprises a wide spectrum of cardiac malformations, including hypoplasia or atresia (abnormal opening or failure of a structure to be tubular) of the aortic and mitral valves and hypoplasia of the left ventricle and ascending aorta. The great vessels are normally related in this congenital anomaly. HLHS has a reported prevalence of 0.2 per 1000 live births and occurs twice as often in boys as in girls. Left untreated, HLHS is lethal (Kau T. et al., Semin Intervent Radiol. 2007; 24 (2): 141-152).

### Coarctation of the Aorta

Coarctation of the aorta accounts for about 5 to 7% of all congenital heart disease. It is defined as a discrete stenosis in the proximal descending thoracic aorta. Only those with the most severe obstruction (e.g., aortic arch atresia or interruption) or associated cardiac defects invariably present in infancy (Jenkins N. P., Ward C. QJM. 1999; 92:365-371). Most other cases are identified because of a murmur or hypertension found on routine examination. Age at presentation is related to the severity rather than the site of obstruction, as a result of cardiac failure or occasionally cerebrovascular accident, aortic dissection, or endocarditis (Jenkins N. P., Ward C. QJM. 1999; 92:365-371). Aortic coarctation may be subclassified into isolated coarctation, coarctation with ventricular septal defect, and coarctation with complex intracardiac anomalies (Backer C. L. et al. Ann Thorac Surg. 2000; 69: S308-S318). An exceedingly rare congenital anomaly is coarctation of a right aortic arch (Maxey T. S. et al. J Card Surg. 2006; 21:261-263).

### Interrupted Aortic Arch

Interrupted aortic arch is defined as the loss of luminal continuity between the ascending and descending aorta and is associated with a multitude of lesions ranging from isolated ventricular septal defects to complex ones (Kau T. et al. Semin Intervent Radiol. 2007; 24 (2): 141-152). An interrupted aortic arch may be subclassified into anatomical types based on the location of the interruption (Maxcy T. S. et al. J Card Surg. 2006; 21:261-263). Although results have improved, repair of this abnormality is associated with a significant mortality and morbidity (Tchervenkov C. I. et al. Semin Thorac Cardiovasc Surg Pediatr Card Surg Annu. 2005:92-102).

### Patent Ductus Arteriosus

A ductus arteriosus Botalli permits blood flow between the aorta (distal to the left subclavian artery) and the pulmonary artery. In a full-term infant, the ductus usually closes within the first 2 days of life. Persistent patency beyond that point is generally permanent, being two to three times as common in girls as in boys. Most of the cases occur as isolated defects. Typical concomitant findings are left ventricle hypertrophy and pulmonary artery dilation. Persistent ductus arteriosus may also be associated with coarctation of the aorta, transposition of the great vessels, and ventricular septal defect (Campbell M. Br Heart J. 1968; 30:4-13).

### Thyroid Ima Artery

The thyroid ima artery is a collateral vessel feeding the thyroid gland (Wolpert S. M. Radiology 1969; 92:333-334). This vessel occurs in up to 16.9% of the population (Vasovic L et al. Ital J Anat Embryol. 2004; 109:189-197). It may be a branch of the aortic arch between the brachiocephalic and left subclavian arteries. However, more frequently it is a branch of the brachiocephalic artery. A further variant of origin is from the right common carotid artery. In the remaining cases, it may originate from the internal mammary, subclavian, or inferior thyroid arteries (Kadir S. In: Kadir S, editor. Atlas of Normal and Variant Angiographic Anatomy. Philadelphia: WB Saunders; 1991. Regional anatomy of the thoracic aorta. pp. 19-54).

### Aberrant Right Subclavian or Brachiocephalic Artery

The right subclavian artery is the last branch of the aortic arch in approximately 1% of individuals (Richardson J. V. et al. Ann Thorac Surg. 1981; 31:426-432). It courses to the right behind the esophagus in approximately 80% of these cases, between the esophagus and trachea in 15%, and anterior to the trachea or mainstem bronchus in 5% (Kadir S. In: Kadir S, editor. Atlas of Normal and Variant Angiographic Anatomy. Philadelphia: WB Saunders; 1991. Regional anatomy of the thoracic aorta. pp. 19-54).

### Right Aortic Arch 25

Right aortic arch is an uncommon anatomical anomaly that occurs in <0.1% of the population (Cina C. S. et al. J Vasc Surg. 2004; 39:131-139). It results from the persistence of the right fourth branchial arch (Kadir S. In: Kadir S, editor. Atlas of Normal and Variant Angiographic Anatomy. Philadelphia: WB Saunders; 1991. Regional anatomy of the thoracic aorta. pp. 19-54). The most common type is the right aortic arch with an aberrant left subclavian artery. The vessels originate in the following order, left common carotid, right common carotid, right subclavian, and left subclavian artery. This type is rarely associated with congenital heart disease. However, symptoms may arise from vascular ring formation (Son J. A. et al. J Card Surg. 1999; 14:98-102). The mirror-image type (left brachiocephalic trunk, right common carotid and subclavian arteries) is almost always associated with congenital heart disease, especially the cyanotic type (McElhinney D. B. et al. Pediatr Cardiol. 2001; 22:285-291).

### Ductus Diverticulum

The aortic isthmus in adults has a variable appearance. Its configuration may show a concavity, a straightening or slight convexity, or a discrete focal bulge. The latter finding represents a ductus diverticulum, present in about 9% of individuals. Representing the most distal segment of the embryonic right arch, the ductus diverticulum is a fusiform dilation of the ventromedial portion of the proximal descending thoracic aorta. At times a prominent ductus diverticulum may resemble a traumatic pseudoaneurysm of the aortic isthmus (Goodman P. C. et al. Cardiovasc Intervent Radiol. 1982; 5:1-4).

### Double Aortic Arch

The double aortic arch is a rare anomaly caused by persistence (to varying degrees) of the fetal double aortic arch system (Kadir S. In: Kadir S, editor. Atlas of Normal and Variant Angiographic Anatomy. Philadelphia: WB Saunders; 1991. Regional anatomy of the thoracic aorta. pp. 19-54). The ascending aorta divides into two arches that pass to either side of the esophagus and trachea and reunite to form the descending aorta. Therefore, it is a form of complete vascular ring, resulting in noncardiac morbidity, but rarely associated with intracardiac defects (Alsenaidi K. et al. Pediatrics. 2006; 118: e1336-e1341). The descending aorta is usually on the left side. Most commonly, one arch is dominant, whereas the other may be of small caliber or represented by a fibrous band.

### Cervical Aortic Arch

The cervical aortic arch refers to an unusually high location of the aortic arch in the low or midneck region (Kadir S. In: Kadir S, editor. Atlas of Normal and Variant Angiographic Anatomy. Philadelphia: WB Saunders; 1991. Regional anatomy of the thoracic aorta. pp. 19-54). This rare type of aortic arch anomaly is presumed to result from persistence of the third aortic arch and regression of the normal fourth arch. Abnormalities of brachiocephalic arterial branching and arch laterality are common in patients with a cervical aortic arch (McElhinney D. B. et al. Pediatr Cardiol. 2001; 22:285-291). There is no association with congenital heart disease, and the anomaly occurs most frequently in association with a right aortic arch. Most of the patients with this anomaly are asymptomatic, but symptoms of dysphagia and respiratory distress due to the compression by the vascular ring have been reported (Acikel U. et al. Angiology 1997; 48:659-662).

### Bovine Aortic Arch 5

A common brachiocephalic trunk (also known as the innominate artery), in which both common carotid arteries and the right subclavian artery arise from a single trunk off the arch, is the most frequent normal variant of aortic arch branching (Kadir S. In: Kadir S, editor. Atlas of Normal and Variant Angiographic Anatomy. Philadelphia: WB Saunders; 1991. Regional anatomy of the thoracic aorta. pp. 19-54) (FIG. 4). The innominate artery and the left common carotid artery have a common origin. Therefore, only 2 great vessels originate from the aortic arch (Layton K. F. et al. Am J Neuroradiol. 2006; 27:1541-1542). Overall, this pattern of branching is seen in approximately 13% of patients (Lippert H, Pabst R. Aortic arch. In: Arterial Variations in Man: Classification and Frequency. Munich, Germany. J F Bergmann-Verlag; 1985:3-10). Although the term bovine aortic arch is ascribed to this anomaly, it is not commonly found in cattle (Layton K. F. et al. Am J Neuroradiol. 2006; 27:1541-1542).

### Other Variant Branching

Variations in the sequence of branching of the major arch vessels also occur (<0.5%) (Kadir S. In: Kadir S, editor. Atlas of Normal and Variant Angiographic Anatomy. Philadelphia: WB Saunders; 1991. Regional anatomy of the thoracic aorta. pp. 19-54). For example, the left subclavian artery may be the second branch (before the left common carotid), or the internal and external carotid arteries may originate independently from the aortic arch (Nelson M. L., Sparks C. D. Clin Anat. 2001; 14:62-65).

### Variant Origin of Vertebral Arteries

Various unusual vertebral artery origins exist (Yamaki K. et al. Anat Sci Int. 2006; 81:100-106; Konigsberg R. A. et al. Catheter Cardiovasc Interv. 2003; 59:244-250). For example, the left vertebral artery arises from the aortic arch, with reported prevalences of 2.4 to 5.8% (Lemke A. J. et al. Am J Neuroradiol. 1999; 20:1318-1321). The most frequent location is between the left common carotid and subclavian arteries (Kadir S. In: Kadir S, editor. Atlas of Normal and Variant Angiographic Anatomy. Philadelphia: WB Saunders; 1991. Regional anatomy of the thoracic aorta. pp. 19-54). Rarely, the proximal left vertebral artery is duplicated in which one part arises from the arch and the other from the left subclavian, or both originate from the aortic arch. Occasionally, the left vertebral artery is the last branch of the aortic arch, which is rarely true for both vertebral arteries (Goray V. B. et al. Am J Neuroradiol. 2005; 26:93-95).

The existence of aortic and vertebral artery variations inhibits the treatment of diseases that require endovascular intervention via a transfemoral approach. For example, the acute angle at which the left common carotid artery branches from the aortic arch in the bovine arch configuration makes mechanical endovascular neurointervention difficult, especially when additional tortuosity (i.e., twists) in the aorta and/or the carotid artery are present. Currently, catheters exist that can access the origin of the left common carotid artery when arterial variations exist. However, when a wire is advanced through these catheters in order to achieve distal access to the artery head, these catheters lack adequate support which results in kickback into the aortic arch of the advancing wire. The lack of adequate support and the resulting kickback of the advancing wire make effective treatment impossible. Even when catheterization is achieved in these situations, the process of arriving at the correct combination of catheters and wires results in long treatment delays. In cases of acute stroke, long delays in obtaining access to arteries often leads to additional irreversible cell death with additional permanent neurologic injury.

Therefore, a need exists for an endovascular device capable of treating diseases that require endovascular intervention in a patient suffering from a blood vessel anomaly. Described herein are endovascular devices capable of effectively treating such patients by providing support and thus preventing kickback of an advancing wire, resulting in distal blood vessel access, clot retrieval, embolization of an aneurysm and/or embolization of an arteriovenous malformation (AVM).

### SUMMARY OF THE INVENTION

In one aspect, provided herein are endovascular devices comprising: a tube comprising: at least one side-hole; a first segment comprising a primary opening; and a second segment, wherein the first segment extends from the primary opening to the side-hole and the second segment extends from the side-hole and tapers to an end, wherein the side-hole and the first segment form a working lumen, and wherein the second segment forms a support lumen, wherein the support lumen is effective: to provide stability to the endovascular device; and to prevent kickback of the endovascular device.

In another aspect, provided herein are endovascular devices comprising: a tube comprising: (a) a side-hole; (b) a first segment comprising a primary opening; and (c) a second segment comprising an end that in cross-section is circular, wherein the side-hole divides the endovascular device into the first segment and the second segment; wherein the first segment extends from the primary opening to the side-hole and the second segment extends from the side-hole to an end hole; wherein the side-hole and the first segment form a working lumen, and the second segment forms a support lumen, and wherein the support lumen is effective: (i) to provide stability to the working lumen of the endovascular device; (ii) to anchor the endovascular device within a blood vessel; (iii) to prevent catheter coil prolapse due to a counterforce against the endovascular device by one or more additional endovascular devices being delivered through the endovascular device to a more distal location or by a prolapsed coil tail of the endovascular device; and (iv) to facilitate placement of the one or more additional endovascular devices distally. According to some embodiments, no ramp is disposed within said tube at or near said side-hole. According to some embodiments, the second segment extends from the side-hole and tapers to the end hole.

In another aspect, provided herein are endovascular devices comprising: a tube comprising: (a) a side-hole; (b) a first segment comprising a primary opening; and (c) a second segment comprising an end that in cross-section is circular, wherein the side-hole divides the endovascular device into the first segment and the second segment; wherein said device further comprises a bend between the first segment and the second segment to assist in anchoring the endovascular device; wherein the first segment extends from the primary opening to the side-hole and the second segment extends from the side-hole to an end hole; wherein the side-hole and the first segment form a working lumen, and the second segment forms a support lumen; and wherein the support lumen is effective: (i) to provide stability to the working lumen of the endovascular device; (ii) to anchor the endovascular device within a blood vessel; (iii) to prevent catheter coil prolapse due to a counterforce against the endovascular device by one or more additional endovascular devices being delivered through the endovascular device to a more distal location or by a prolapsed coil tail of the endovascular device; and (iv) to facilitate placement of the one or more additional endovascular devices distally. According to some embodiments, no ramp is disposed within said tube at or near said side-hole. According to some embodiments, the second segment extends from the side-hole and tapers externally to the end hole.

In another aspect, provided herein are endovascular devices comprising: a tube comprising: (a) a side-hole; (b) a first segment comprising a primary opening; and (c) a second segment comprising an end that in cross-section is circular, wherein no ramp is disposed within the tube, wherein the side-hole divides the endovascular device into the first segment and the second segment; wherein the first segment extends from the primary opening to the side-hole and the second segment extends from the side-hole to an end hole; wherein the side-hole and the first segment form a working lumen, and the second segment forms a support lumen; and wherein the support lumen is effective: (i) to provide stability to the working lumen of the endovascular device; (ii) to anchor the endovascular device within a blood vessel; (iii) to prevent catheter coil prolapse due to a counterforce against the endovascular device by one or more additional endovascular devices being delivered through the endovascular device to a more distal location or by a prolapsed coil tail of the endovascular device; and (iv) to facilitate placement of the one or more additional endovascular devices distally. According to some embodiments, the second segment extends from the side-hole and tapers to the end hole.

In another aspect, provided herein are endovascular devices comprising: a tube comprising (a) a side-hole; (b) a first segment comprising a primary opening; and (c) a second segment comprising an end that in cross-section is circular, wherein the side-hole divides the endovascular device into the first segment and the second segment; wherein the first segment extends from the primary opening to the side-hole and the second segment extends from the side-hole and tapers externally to an end hole; wherein the side-hole and the first segment form a working lumen, and the second segment forms a support lumen; and wherein the support lumen is effective: (i) to provide stability to the working lumen of the endovascular device; (ii) to anchor the endovascular device within a blood vessel; (iii) to prevent catheter or other device prolapse due to a counterforce against the endovascular device by one or more additional endovascular devices being delivered through the endovascular device to a more distal location or by a prolapsed coil tail of the endovascular device; and (iv) to facilitate placement of the one or more additional endovascular devices distally. According to some embodiments, no ramp is disposed within said tube at or near said side-hole. According to some embodiments, the second segment maintains a constant inner lumen diameter that does not substantially decrease at a distal end.

In another aspect, provided herein are endovascular devices comprising: a tube comprising (a) a side-hole; (b) a first segment comprising a primary opening; and (c) a second segment comprising an end that in cross-section is circular, wherein the side-hole divides the endovascular device into the first segment and the second segment; wherein the first segment extends from the primary opening to the side-hole and the second segment extends from the side-hole to an end hole and externally maintains a constant outer diameter that does not substantially decrease at a distal end; wherein the side-hole and the first segment form a working lumen, and the second segment forms a support lumen; and wherein the support lumen is effective: (i) to provide stability to the working lumen of the endovascular device; (ii) to anchor the endovascular device within a blood vessel; (iii) to prevent catheter or other device prolapse due to a counterforce against the endovascular device by one or more additional endovascular devices being delivered through the endovascular device to a more distal location or by a prolapsed coil tail of the endovascular device; and (iv) to facilitate placement of the one or more additional endovascular devices distally. According to some embodiments, no ramp is disposed within said tube at or near the side-hole.

In another aspect, provided herein are endovascular devices comprising: a tube comprising: (a) a side-hole segment comprising multiple side-holes; (b) a first segment comprising a primary opening; and (c) a second segment comprising an end, wherein said multiple side-holes comprise: (A) a first (proximal) side-hole that is the side-hole most proximal to the proximal end of the tube and having a proximal side and a distal side; and (B) a second (distal) side-hole that is the side-hole most distal to the distal end of the tube and having a proximal side and a distal side, wherein the first segment extends from the primary opening to the proximal side of the first side-hole and the second segment extends from the distal side of the second side-hole to an end hole; wherein the side-hole segment extends from the proximal side of the first side-hole to the distal side of the second side-hole; wherein the side-hole segment and the first segment form a working lumen, and the second segment forms a support lumen, and wherein the support lumen is effective: (i) to provide stability to the working lumen of the endovascular device; (ii) to anchor the endovascular device within a blood vessel; (iii) to prevent catheter coil prolapse due to a counterforce against the endovascular device by one or more additional endovascular devices being delivered through the endovascular device to a more distal location or by a prolapsed coil tail of the endovascular device; and (iv) to facilitate placement of the one or more additional endovascular devices distally. According to some embodiments, no ramp is disposed within said tube at or near the side-holes. According to some embodiments, the second segment extends from the distal side of the second side-hole and tapers to the end hole. According to some embodiments, the end of the second segment is circular in cross-section. According to some embodiments, the end of the second segment is oval or flattened in cross-section. According to some embodiments, the multiple side-holes are along the same length of said tube, at different circumferential locations. According to some embodiments, the multiple side-holes are staggered at different lengths along said tube, either along the same or various circumferential locations. According to some embodiments, some of the side-holes are staggered at different lengths along said tube and some are along the same length. According to some embodiments, side-holes may be long partially overlapping lengths of said tube, along varying circumferential locations.

In another aspect, provided herein are endovascular devices comprising: a tube comprising: (a) a side-hole segment comprising multiple side-holes; (b) a first segment comprising a primary opening; and (c) a second segment comprising an end, wherein said multiple side-holes comprise: (A) a first (proximal) side-hole that is the side-hole most proximal to the proximal end of the tube and having a proximal side and a distal side; and (B) a second (distal) side-hole that is the side-hole most distal to the distal end of the tube and having a proximal side and a distal side, wherein said device further comprises a bend between the first segment and the second segment to assist in anchoring the endovascular device; wherein the first segment extends from the primary opening to the proximal side of the first side-hole and the second segment extends from the distal side of the second side-hole to an end hole; wherein the side-hole segment and the first segment form a working lumen, and the second segment forms a support lumen; and wherein the support lumen is effective: (i) to provide stability to the working lumen of the endovascular device; (ii) to anchor the endovascular device within a blood vessel; (iii) to prevent catheter coil prolapse due to a counterforce against the endovascular device by one or more additional endovascular devices being delivered through the endovascular device to a more distal location or by a prolapsed coil tail of the endovascular device; and (iv) to facilitate placement of the one or more additional endovascular devices distally. According to some embodiments, no ramp is disposed within said tube at or near the side-holes. According to some embodiments, the end of the second segment is circular in cross-section. According to some embodiments, the end of the second segment is oval or flattened in cross-section. According to some embodiments, the second segment extends from the distal side of the second side-hole and tapers externally to the end hole. According to some embodiments, the multiple side-holes are along the same length of said tube, at different circumferential locations. According to some embodiments, the multiple side-holes are staggered at different lengths along said tube, either along the same or various circumferential locations. According to some embodiments, some of the side-holes are staggered at different lengths along said tube and some are along the same length. According to some embodiments, side-holes may be long partially overlapping lengths of said tube, along varying circumferential locations.

In another aspect, provided herein are endovascular devices comprising: a tube comprising: (a) a side-hole segment comprising multiple side-holes; (b) a first segment comprising a primary opening; and (c) a second segment comprising an end, wherein said multiple side-holes comprise: (A) a first (proximal) side-hole that is the side-hole most proximal to the proximal end of the tube and having a proximal side and a distal side; and (B) a second (distal) side-hole that is the side-hole most distal to the distal end of the tube and having a proximal side and a distal side, wherein no ramp is disposed within the tube, wherein the first segment extends from the primary opening to the proximal side of the first side-hole and the second segment extends from the distal side of the second side-hole to an end hole; wherein the side-hole segment extends from the proximal side of the first side-hole to the distal side of the second side-hole; wherein the side-hole segment and the first segment form a working lumen, and the second segment forms a support lumen; and wherein the support lumen is effective: (i) to provide stability to the working lumen of the endovascular device; (ii) to anchor the endovascular device within a blood vessel; (iii) to prevent catheter coil prolapse due to a counterforce against the endovascular device by one or more additional endovascular devices being delivered through the endovascular device to a more distal location or by a prolapsed coil tail of the endovascular device; and (iv) to facilitate placement of the one or more additional endovascular devices distally. According to some embodiments, the second segment extends from the distal side of the second side-hole and tapers to the end hole. According to some embodiments, the end of the second segment is circular in cross-section. According to some embodiments, the end of the second segment is oval or flattened in cross-section. According to some embodiments, the multiple side-holes are along the same length of said tube, at different circumferential locations. According to some embodiments, the multiple side-holes are staggered at different lengths along said tube, either along the same or various circumferential locations. According to some embodiments, some of the side-holes are staggered at different lengths along said tube and some are along the same length. According to some embodiments, side-holes may be long partially overlapping lengths of said tube, along varying circumferential locations.

In another aspect, provided herein are endovascular devices comprising: a tube comprising (a) a side-hole segment comprising multiple side-holes; (b) a first segment comprising a primary opening; and (c) a second segment comprising an end, wherein said multiple side-holes comprise: (A) a first (proximal) side-hole that is the side-hole most proximal to the proximal end of the tube and having a proximal side and a distal side; and (B) a second (distal) side-hole that is the side-hole most distal to the distal end of the tube and having a proximal side and a distal side, wherein the first segment extends from the primary opening to the proximal side of the first side-hole and the second segment extends from the distal side of the second side-hole and tapers externally to an end hole; wherein the side-hole segment extends from the proximal side of the first side-hole to the distal side of the second side-hole; wherein the side-hole segment and the first segment form a working lumen, and the second segment forms a support lumen; and wherein the support lumen is effective: (i) to provide stability to the working lumen of the endovascular device; (ii) to anchor the endovascular device within a blood vessel; (iii) to prevent catheter or other device prolapse due to a counterforce against the endovascular device by one or more additional endovascular devices being delivered through the endovascular device to a more distal location or by a prolapsed coil tail of the endovascular device; and (iv) to facilitate placement of the one or more additional endovascular devices distally. According to some embodiments, no ramp is disposed within the tube at or near the side-holes. According to some embodiments, the end of the second segment is circular in cross-section. According to some embodiments, the end of the second segment is oval or flattened in cross-section. According to some embodiments, the second segment maintains a constant inner lumen diameter that does not substantially decrease at a distal end. According to some embodiments, the multiple side-holes are along the same length of said tube, at different circumferential locations. According to some embodiments, the multiple side-holes are staggered at different lengths along said tube, either along the same or various circumferential locations. According to some embodiments, some of the side-holes are staggered at different lengths along said tube and some are along the same length. According to some embodiments, side-holes may be long partially overlapping lengths of said tube, along varying circumferential locations.

In another aspect, provided herein are endovascular devices comprising: a tube comprising (a) a side-hole segment comprising multiple side-holes; (b) a first segment comprising a primary opening; and (c) a second segment comprising an end, wherein said multiple side-holes comprise: (A) a first (proximal) side-hole that is the side-hole most proximal to the proximal end of the tube and having a proximal side and a distal side; and (B) a second (distal) side-hole that is the side-hole most distal to the distal end of the tube and having a proximal side and a distal side, wherein the first segment extends from the primary opening to the proximal side of the first side-hole and the second segment extends from the distal side of the second side-hole to an end hole and externally maintains a constant outer diameter that does not substantially decrease at a distal end; wherein the side-hole segment extends from the proximal side of the first side-hole to the distal side of the second side-hole; wherein the side-hole segment and the first segment form a working lumen, and the second segment forms a support lumen; and wherein the support lumen is effective: (i) to provide stability to the working lumen of the endovascular device; (ii) to anchor the endovascular device within a blood vessel; (iii) to prevent catheter or other device prolapse due to a counterforce against the endovascular device by one or more additional endovascular devices being delivered through the endovascular device to a more distal location or by a prolapsed coil tail of the endovascular device; and (iv) to facilitate placement of the one or more additional endovascular devices distally. According to some embodiments, no ramp is disposed within the tube at or near the side-holes. According to some embodiments, the end of the second segment is circular in cross-section. According to some embodiments, the multiple side-holes are along the same length of said tube, at different circumferential locations. According to some embodiments, the multiple side-holes are staggered at different lengths along said tube, either along the same or various circumferential locations. According to some embodiments, some of the side-holes are staggered at different lengths along said tube and some are along the same length. According to some embodiments, side-holes may be long partially overlapping lengths of said tube, along varying circumferential locations.

In another aspect, provided herein are methods of using endovascular devices described herein, in a patient for a desired procedure or intervention (e.g., a neuro-intervention), utilizing standard endovascular techniques under fluoroscopic guidance, comprising the steps of: (a) obtaining femoral arterial access; (b) introducing a diagnostic catheter into the origin of an innominate artery associated with a bovine origin left common carotid artery; (c) advancing an exchange length wire in a right subclavian artery, then into at least one of a right axillary artery and a right brachial artery; (d) removing said diagnostic catheter, and leaving said exchange length wire in place; (c) advancing the endovascular device over said wire, and a distal end hole into the axillary artery, or more distally into the brachial artery, and positioning said side-hole into the innominate artery substantially adjacent to the origin of the left common carotid artery; (f) rotating the endovascular device so that said side-hole faces the origin of the left common carotid artery; (g) removing said exchange length wire while leaving the endovascular device in place; (h) advancing a guide catheter through the endovascular device, over a second wire, out said side-hole, and into the left internal carotid artery, while said distal segment of the endovascular device remains in the right axillary artery and serves to anchor the endovascular device in place to prevent kickback and prolapse of all devices into the aorta while a guide catheter is advanced into position; (i) removing said second wire; (j) advancing at least one of an additional therapeutic catheter and a device delivery platform into an intracranial circulation; (k) after completing the desired procedure or intervention, removing said at least one of the additional therapeutic catheter and device delivery platform; (l) removing said guide catheter; and (m) removing the endovascular device and obtaining femoral hemostasis.

In another aspect, provided herein are methods of using an endovascular device described herein, in a patient with a target side-wall intracranial aneurysm, utilizing standard endovascular techniques under fluoroscopic guidance, comprising the steps of: (a) introducing a guide catheter, via standard endovascular techniques, into a precerebral artery, including at least one of a vertebral artery and an internal carotid artery, in the same circulation as the target intracranial aneurysm; (b) advancing the endovascular device through said guide catheter, over at least one of a delivery wire and an additional catheter, until said end-hole is distal to said target aneurysm and said side-hole is substantially adjacent to the center of the neck of said target aneurysm; (c) rotating the endovascular device so that said side-hole optimally faces the center of the neck of the target aneurysm; (d) removing said at least one of said delivery wire and said additional catheter; (c) advancing an additional microcatheter over a second wire, through the first segment of the endovascular device, and into the center of the target aneurysm, while leaving said second segment in position, said second segment of the endovascular device serves to anchor the endovascular device in place, to prevent kickback and prolapse of all endovascular devices while said additional catheters and endovascular devices are advanced into position; (f) removing said second wire; (g) pushing a single aneurysm treatment device, or multiple devices sequentially, through said additional microcatheter into position within said target aneurysm, attached proximally to a delivery system for said aneurysm treatment device(s), deploying said aneurysm treatment device(s), and detaching said aneurysm treatment device or devices from said delivery system after radiographic confirmation of positioning; (h) removing said delivery system for said aneurysm treatment device(s), (i) subsequently removing said additional microcatheter; (j) removing the endovascular device; and (k) removing said guide catheter and obtaining appropriate hemostasis at a vascular access site.

In another aspect, provided herein are methods of using an endovascular device with a side-hole segment comprising multiple side-holes described herein, in a patient with a target side-wall intracranial aneurysm, utilizing standard endovascular techniques under fluoroscopic guidance, comprising the steps of: (a) introducing a guide catheter, via standard endovascular techniques, into a precerebral artery, including at least one of a vertebral artery and an internal carotid artery, in the same circulation as the target intracranial aneurysm; (b) advancing the endovascular device through said guide catheter, over at least one of a delivery wire and an additional catheter, until said end-hole is distal to said target aneurysm and said side-hole is substantially adjacent to the center of the neck of said target aneurysm; (c) confirming via imaging which side-hole is centered on said target aneurysm; (d) removing said at least one of said delivery wire and said additional catheter; (c) advancing an additional microcatheter over a second wire, through the first segment of the endovascular device, through the side hole positioned adjacent to the center of the neck of the target aneurysm and into the center of the target aneurysm, while leaving said second segment in position, said second segment of the endovascular device serves to anchor the endovascular device in place, to prevent kickback and prolapse of all endovascular devices while said additional catheters and endovascular devices are advanced into position; (f) removing said second wire; (g) pushing a single aneurysm treatment device, or multiple devices sequentially, through said additional microcatheter into position within said target aneurysm, attached proximally to a delivery system for said aneurysm treatment device(s), deploying said aneurysm treatment device(s), and detaching said aneurysm treatment device or devices from said delivery system after radiographic confirmation of positioning; (h) removing said delivery system for said aneurysm treatment device(s), (i) subsequently removing said additional microcatheter; (j) removing the endovascular device; and (k) removing said guide catheter and obtaining appropriate hemostasis at a vascular access site.

According to one embodiment, the length of the first segment ranges from about 50 cm to about 100 cm. According to some embodiments, the length of the first segment ranges from about 100 cm to about 170 cm.

According to one embodiment, the second segment extends from about 20 cm to about 60 cm in length from the side-hole. According to some embodiments, the second segment extends from about 1 cm to about 20 cm in length from the side-hole.

According to some embodiments, the internal diameter of the working lumen ranges from about 0.0254 cm (0.0100 inches) to about 26 Fr (0.3410 inches). According to some embodiments, the internal diameter of the working lumen ranges from about 4 Fr (0.0530 inches) to about 12 Fr (0.1580 inches). According to some embodiments, the internal diameter of the second segment ranges from about 0.0020 cm (0.0008 inches) to about 23 Fr (0.3018 inches).

According to some embodiments, the endovascular device comprises a catheter, a wire, a therapeutic balloon, an embolic device, a therapeutic stent, or another endovascular device, as well as combinations thereof. According to some embodiments, the endovascular device further comprises an angled extension that extends out of a side-hole. The angle of the angled extension ranges from about 10 degrees to about 180 degrees, relative to the primary tube. According to some embodiments, the angled extension comprises a shape memory polymer (SMP), a shape memory alloy (SMA) or a combination thereof. According to some embodiments, the endovascular device comprises an actively adjustable angled extension extending from the side-hole serving to facilitate steering of the one or more additional devices into said blood vessel.

According to some embodiments, the endovascular device further comprises an introducer.

According to some embodiments, the blood vessel comprises an anatomical variation comprising an acute angulation. According to some embodiments, the acute angulation is an aortic arch variation. According to some embodiments, the acute angulation is a vertebral artery variation. According to some embodiments, the aortic arch variation is bovine arch variation.

According to some embodiments, the blood vessel is accessed through the femoral artery or through the brachial artery or through the radial artery or through the carotid artery.

According to some embodiments, the working lumen is a conduit through which one or more additional endovascular devices is advanced into a blood vessel.

According to some embodiments, the one or more additional endovascular devices comprises a catheter, a wire, a therapeutic balloon, an embolic device, a therapeutic stent, or another endovascular device, as well as combinations thereof. In many procedures there may be multiple additional catheters, wires, balloons, and/or other endovascular devices that may be advanced through the primary catheter, either side-by side, or in a progressive telescoping fashion within each other.

According to some embodiments, the side-hole comprises an angled extension. According to some embodiments, the angle of the angled extension ranges from about 0 degrees to about 359 degrees. According to some embodiments, the angle of the angled extension is fixed. According to some embodiments, the angle of the angled extension is adjustable.

According to some embodiments, the endovascular device comprises a luer lock. According to some embodiments, the length of the first segment ranges from about 10 cm to about 130 cm. According to some embodiments, the length of the device ranges from about 10 cm to about 520 cm. According to some embodiments, the length of the second segment ranges from about 1% to about 300% of the length of the first segment.

According to some embodiments, the support lumen is of an “S” shape. According to some embodiments, the “S” shape is a shepherd's hook shape. According to some embodiments, length of the support lumen is greater than 4% longer than length of the working lumen.

According to some embodiments, the support lumen is circular in cross section. According to some embodiments, the support lumen is oval in cross section. According to some embodiments, the support lumen is flattened in cross section. According to some embodiments, the support lumen maintains a constant inner lumen diameter. According to some embodiments, the support lumen has an inner lumen diameter that varies along the course of the endovascular device.

According to some embodiments, the endovascular device further comprises at least one balloon disposed thereon, and at least one additional lumen substantially within the wall of the intravascular portion of said device that serves solely to inflate and deflate said at least one balloon.

These and other advantages of the invention will be apparent to those of ordinary skill in the art by reference to the following detailed description and the accompanying drawings.

### BRIEF DESCRIPTION OF THE DRAWINGS

FIG. 1 shows an illustration of a side view of one embodiment of a dual lumen catheter of the described invention.

15

FIG. 2 shows an illustration of a cross-sectional view of one embodiment of a dual lumen catheter of the described invention.

FIG. 3 shows an illustration of a side view of one embodiment of a single lumen catheter of the described invention.

FIG. 4 shows an illustration of the most common aortic arch branching pattern found in humans (from Layton K. F. et al., Am J Neuroradiol. 2006; 27:141-1542).

FIG. 5 shows an illustration of the aortic arch branching pattern in bovine arch variation (from Layton K. F. et al. Am J Neuroradiol. 2006; 27:141-1542).

FIG. 6 shows an illustration of a side view of one embodiment of a support lumen of the invention.

FIG. 7 shows an illustration of one embodiment of the endovascular device comprising an “S”-shape support lumen inserted into an aortic arch.

FIG. 8 shows an illustrative view of the cerebral arteries.

FIG. 9 shows an illustrative view of the cerebral arteries. (from Netter F. H. The CIBA Collection of Medical Illustrations: Volumes 1, Nervous System. Vol. 1. Part I. CIBA: USA. 1986. pp. 256).

FIG. 10 shows an illustration of one embodiment of the endovascular device inserted in the left middle cerebral artery (L-MCA) with the side-hole oriented to access an acutely angled branch feeder of an arteriovenous malformation (AVM).

FIG. 11 shows an illustration of one embodiment of the endovascular device is depicted inserted in the left middle cerebral artery (L-MCA) with the side-hole oriented to access an acutely angled branch feeder of an arteriovenous malformation (AVM) and with a catheter being advanced out of the side-hole and into the branch feeder of the AVM.

FIG. 12 shows an illustrative embodiment of an endovascular device according to the described invention. 1310 is an illustration of inflatable balloons attached around the circumference of the endovascular device located proximal to the side-hole (310), (1470).

FIG. 13A and FIG. 13B show an illustrative embodiment of an endovascular device according to the invention. 1410 is an illustration of a luer lock. 1420 identifies the beginning of the outer catheter. 1430 is a general illustration of grips. 1431 is an illustration of grips on the inner catheter. 1432 is an illustration of grips on the outer catheter. 1440 is an illustration of an outer catheter. Said grips can facilitate rotation of the inner catheter to properly align the side hole in a desired orientation. 1450 is an illustration of an angled extension protruding from the side-hole (1470) positioned at the base of an aneurysm; length of the angled extension is less than the length of the side-hole (1470). 1460 is an illustration of an inner catheter of the endovascular device. 1480 is an illustration of the distal support segment of the endovascular device. In some alternative embodiments, this additional extension (1450) can rather be an additional catheter advanced through the primary catheter and the side hole, which serves to not limit the length.

FIG. 14 shows an illustrative embodiment of an endovascular device (1400) according to the invention. This embodiment shows no distal support segment. 1310 illustrates an array of inflatable balloons attached around the circumference of the endovascular device.

FIG. 15 shows a cross-sectional view of some embodiments of a catheter of the invention.

FIG. 16 shows a cross-sectional view of some embodiments of a catheter of the invention.

### DETAILED DESCRIPTION OF THE INVENTION

### Glossary

The term “ablation” as used herein, refers to a procedure that uses radiofrequency energy (e.g., microwave heat) to destroy a small area of heart tissue that is causing rapid and irregular heartbeats. Destroying this tissue restores the hearts regular rhythm. The procedure is also called radiofrequency ablation.

The terms “acute angle” and “acute angulation” are used interchangeably herein to refer to a sharp, obstructive or abnormal angle or bend (e.g., less than 90 degrees) in an organ, artery, vessel, etc.

### Anatomical Terms

When referring to animals that typically have one end with a head and mouth, with the opposite end often having the anus and tail, the head end is referred to as the cranial end, while the tail end is referred to as the caudal end. Within the head itself, rostral refers to the direction toward the end of the nose, and caudal is used to refer to the tail direction. The surface or side of an animal's body that is normally oriented upwards, away from the pull of gravity, is the dorsal side; the opposite side, typically the one closest to the ground when walking on all legs, swimming or flying, is the ventral side. On the limbs or other appendages, a point closer to the main body is “proximal”; a point farther away is “distal”. Three basic reference planes are used in zoological anatomy. A “sagittal” plane divides the body into left and right portions. The “midsagittal” plane is in the midline, i.e. it would pass through midline structures such as the spine, and all other sagittal planes are parallel to it. A “coronal” plane divides the body into dorsal and ventral portions. A “transverse” plane divides the body into cranial and caudal portions.

When referring to humans, the body and its parts are always described using the assumption that the body is standing upright. Portions of the body which are closer to the head end are “superior” (corresponding to cranial in animals), while those farther away are “inferior” (corresponding to caudal in animals). Objects near the front of the body are referred to as “anterior” (corresponding to ventral in animals); those near the rear of the body are referred to as “posterior” (corresponding to dorsal in animals). A transverse, axial, or horizontal plane is an X-Y plane, parallel to the ground, which separates the superior/head from the inferior/feet. A coronal or frontal plane is an Y-Z plane, perpendicular to the ground, which separates the anterior from the posterior. A sagittal plane is an X-Z plane, perpendicular to the ground and to the coronal plane, which separates left from right. The midsagittal plane is the specific sagittal plane that is exactly in the middle of the body.

Structures near the midline are called medial and those near the sides of animals are called lateral. Therefore, medial structures are closer to the midsagittal plane, lateral structures are further from the midsagittal plane. Structures in the midline of the body are median. For example, the tip of a human subject's nose is in the median line.

Ipsilateral means on the same side, contralateral means on the other side and bilateral means on both sides. Structures that are close to the center of the body are proximal or central, while ones more distant are distal or peripheral. For example, the hands are at the distal end of the arms, while the shoulders are at the proximal ends.

The terms “anomaly”, “variation”, “abnormality” and “aberration” are used interchangeably herein to refer to a deviation from what is standard, normal or expected. For example, “bovine arch variation” is an anatomical deviation from the most common aortic arch branching pattern in humans. By way of additional example, an anomaly can occur in a blood vessel having tortuosity.

The term “aneurysm”, as used herein, refers to a localized widening (dilatation) of an artery, a vein, or the heart. At the point of an aneurysm, there is typically a bulge, where the wall of the blood vessel or organ is weakened and may rupture.

Blood flow in most aneurysms is regular and predictable primarily according to the geometric relationship between the aneurysm and its parent artery. As blood flows within the parent artery with an aneurysm, divergence of blood flow, as occurs at the inlet of the aneurysm, leads to dynamic disturbances, producing increased lateral pressure and retrograde vortices that are easily converted to turbulence. Blood flow proceeds from the parent vessel into the aneurysm at the distal 20 or downstream extent of the aneurysm neck (i.e., the transition from the sac to the parent artery), circulates around the periphery along the aneurysm wall from the neck to the top of the fundus (i.e., aneurysm sac) (downstream to upstream), returning in a type of “isotropic shower” along the aneurysm wall toward the neck region, and exits the closest extent of the aneurysm neck into the parent vessel (See, e.g., Strother C. M. Neuroradiology 1994; 36:530-536; Moulder P. V. 25 Physiology and biomechanics of aneurysms. In: Kerstein M D, Moulder P V, Webb W R, cds. Aneurysms. Baltimore, Md.: Williams & Wilkins; 1983:20).

As flow persists, areas of stagnation or vortices develop within a central zone of the aneurysm. These rotating vortices, formed at the entrance to the aneurysm at each systole (i.e., ventricle contraction) and then circulated around the aneurysm, are caused by the slipstreams or regions of recirculating flow rolling upon themselves when they enter the aneurysm at its downstream wall during systole. The stagnant vortex zone occurs in the center and at the fundus or upper portion of the aneurysm and becomes more pronounced in larger aneurysms. It is this stagnant zone that is believed to promote the formation of thrombi or blood clots, particularly in giant aneurysms (See, e.g., Gobin Y. P. et al. Neuroradiology 1994; 36:530-536; Hademenos G. J. and Massoud T. F. Stroke 1997; 28:2067-2077).

The term “abdominal aortic aneurysm” or “AAA”, as used herein refers to an aortic diameter at least one and one-half times the normal diameter at the level of the renal arteries, which is approximately 2.0 cm. Generally, a segment of abdominal aorta with a diameter of greater than 3.0 cm is considered an aortic aneurysm. Aortic aneurysms constitute the 14th leading cause of death in the United States. Risk factors associated with AAA include age, sex, ethnicity, smoking, hypertension and atherosclerosis, among others (See, e.g., Aggarwal S. et al. Exp Clin Cardiol. 2011; 16 (1): 11-15; Ouriel K. et al. J Vasc Surg. 1992; 15:12-18; Silverberg E. et al. C A Cancer J Clin. 1990; 40:9-26).

The term “arteriovenous malformation” (“AVM”), as used herein, refers to a tangle of abnormal and poorly formed blood vessels (e.g., arteries and veins) which have a higher than normal rate of bleeding compared to normal blood vessels.

AVMs are congenital vascular lesions that occur as a result of capillary mal-development between the arterial and venous systems. Approximately 0.14% of the United States population has an intracranial AVM that poses a significant risk and represents a major life threat, particularly to persons under the age of 50 years. The vessels constituting the AVM are weak and enlarged and serve as direct shunts for blood flow between the high-pressure arterial system and the low-pressure venous system, corresponding to a large pressure gradient and small vascular resistance. The abnormal low-resistance, high-flow shunting of blood within the brain AVM without an intervening capillary bed causes the fragile dilated vessels in the nidus (i.e., tangle of blood vessels) to become structurally abnormal and fatigued, to further enlarge, and to rupture (See, e.g., Wilkins R. H. Neurosurgery 1985; 16:421-430; Graves V. B. et al. Invest Radiol. 1990; 25:952-960; Hademenos G. J. et al., Neurosurgery 1996; 38:1005-1015).

The abnormal microvessels of an AVM serve as passive conduits for blood flow from the arterial circulation directly to the venous circulation, by-passing their normal physiological function of brain tissue perfusion. The hemodynamic consequences of an AVM occur as a result of two interdependent circulatory mechanisms involved in the shunting of blood between artery and vein (See, e.g., Hademenos G. J. and Massoud T. F. Stroke 1996; 27:1072-1083).

In the normal cerebral circulation, blood flows under a high cerebrovascular resistance and high cerebral perfusion pressure. However, the presence of a brain AVM in the normal circulation introduces a second abnormal circuit of cerebral blood flow where the blood flow is continuously shunted under a high perfusion pressure through the AVM, possessing a low cerebrovascular resistance and low venous pressure. The clinical consequence of the abnormal shunt is a significant increase in blood returning to the heart (approximately 4 to 5 times the original amount, depending on the diameter and size of the shunt), resulting in a dangerous overload of the heart and cardiac failure. Volumetric blood flow through an AVM ranges from 200 mL/min to 800 mL/min and increases according to nidus size (See, e.g., Yamada S. Neurol Res. 1993; 15:379-383).

The abnormal shunting of blood flow by brain AVMs rapidly removes or “steals” blood from the normal cerebral circulation and substantially reduces the volume of blood reaching the surrounding normal brain tissue. This phenomenon, known as cerebrovascular steal, depends on the size of the AVM and is the most plausible explanation for the development of progressive neurological deficits. Cerebrovascular steal could translate into additional neurological complications developed as a result of cerebral ischemia or stroke in neuronal territories adjacent to an AVM (See, e.g., Manchola I. F. et al. Neurosurgery 1993; 33:556-562; Hademenos G. J. and Massoud T. F. Stroke 1997; 28:2067-2077).

The term “atherectomy”, as used herein, refers to a minimally invasive endovascular surgery technique for removing atherosclerosis from blood vessels within the body by cutting plaque from the walls of a blood vessel.

The term “atherosclerosis” (also known as “hardening of the arteries”), as used herein, refers to a pathological process in which calcified lipid or fatty deposits from flowing blood accumulate along the innermost intimal layer of a vessel wall. Atherosclerotic plaques are found almost exclusively at the outer wall of one or both daughter vessels at major arterial bifurcations, including the carotid. Atherosclerosis and the development of arterial plaques are the products of a host of independent biochemical processes including the oxidation of low-density lipoproteins, formation of fatty streaks, and the proliferation of smooth muscle cells. As the plaques form, the walls become thick, fibrotic, and calcified. As a result, the lumen narrows, reducing the flow of blood to the tissues supplied by the artery (See, e.g., Hademenos G. J. and Massoud T. F. Stroke 1997; 28:2067-2077; Hademenos G. J. Am Scientist 1997; 85:226-235; Woolf N., Davies M. J. Sci Am Science & Medicine 1994; 1:38-47).

Atherosclerotic deposits promote the development of blood clots or the process of thrombosis, due in part, to flow obstruction and to high shear stresses exerted on the vessel wall by the blood. High wall shear stress mechanically damages the inner wall of the artery, initiating a lesion. Low wall shear stress encourages the deposition of particles on the artery wall, promoting the accumulation of plaque. Turbulence has also been implicated in atherosclerotic disease because it can increase the kinetic energy deposited in the vessel walls and because it can lead to areas of stasis, or stagnant blood flow, that promote dotting. The presence of atherosclerotic lesions introduces an irregular vessel surface, resulting in turbulent blood flow, thus causing the dislodgment of plaques of varying size into the bloodstream. Subsequently, the dislodged plaque lodges into a vessel of smaller size, preventing further passage of blood flow (See, e.g., Hademenos G. J. and Massoud T. F. Stroke 1997; 28:2067-2077).

The term “atresia”, as used herein, refers to the absence or abnormal narrowing of an opening or passage in the body. For example, aortic atresia refers to a rare congenital anomaly in which the aortic orifice is absent or closed.

The term “atrial fibrillation”, as used herein, refers to an irregular and often fast heart rate which may cause symptoms such as heart palpitations, fatigue, and shortness of breath. Atrial fibrillation weakens the cardiac wall and introduces abnormalities in the physiological function of the heartbeat, which ultimately result in reduced systemic pressure, conditions of ischemia and stroke.

The term “brachiocephalic trunk”, also known as “innominate artery”, as used herein, refers to a major vessel that supplies the head, neck and right arm. It is the first of three main branches of the aortic arch, which originates from the upward convexity. After arising in the midline, it courses upwards to the right, crossing the trachea, and bifurcates posteriorly to the right sternoclavicular joint into the right subclavian and right common carotid arteries. It typically measures 4-5 cm in length with a diameter of approximately 12 mm.

The term “brain aneurysm”, as used herein, refers to a cerebrovascular disease that manifests as a pouching or ballooning of the vessel wall (i.e., vascular dilation). The vascular dilatation develops at a diseased site along the arterial wall into a distended sac of stressed and thinned arterial tissue. The fully developed cerebral aneurysm typically ranges in size from a few millimeters to 15 mm but can attain sizes greater than 2.5 cm. If left untreated, the aneurysm may continue to expand until it ruptures, causing hemorrhage, severe neurological complications and deficits, and possibly death (Hademenos G. J. and Massoud T. F. Stroke 1997; 28:2067-2077; Hademenos G. J. Phys Today 1995; 48:24-30).

The two main treatment options for a patient suffering from a brain aneurysm are (i) surgical clipping; and (ii) endovascular coiling. Surgical clipping is an intracranial procedure in which a small metallic clip is placed along the neck of the aneurysm. The clip prevents blood from entering into the aneurysm sac so that it no longer poses a risk for bleeding. The clip remains in place, causing the aneurysm to shrink and permanently scar. Endovascular coiling is a minimally invasive technique in which a catheter is inserted into the femoral artery and navigated through the blood vessels to the vessels of the brain and into the aneurysm. Coils are then packed into the aneurysm to the point where it arises from the blood vessel, thus preventing blood flow from entering the aneurysm. Additional devices, such as a stent or balloon, for example, may be needed to keep the coils in place.

The FDA recently approved the WEB® Embolization System (Microvention Inc., CA) as an alternative treatment for intracranial aneurysms. The WEB® device is an intrasaccular flow disruptor for aneurysm embolization and is based on MICROBRAID™ technology, a dense mesh constructed from a large number of extremely fine wires. When placed inside the aneurysm sac, the WEB® device's mesh bridges the aneurysm neck, disrupting blood flow, and creates a scaffold for enduring treatment.

The term “branch”, as used herein, refers to something that extends from or enters into a main body or source; a division or offshoot from a main stem (e.g., blood vessels); one of the primary divisions of a blood vessel.

The term “coarctation” or “coarctation of the aorta”, as used herein, refers to a congenital narrowing of a short section of the aorta.

The terms “compound curves” and “multi-curves” are used interchangeably herein to refer to multiple deflection points along the length of a catheter. By way of example, two deflection points allow a catheter to be deflected into an “S” shape or the shape of a shepherd's hook.

The term “curve diameter”, as used herein, refers to the furthest distance a catheter moves from its straight axis as it is being deflected. The curve diameter does not always remain constant during deflection and does not necessarily indicate the location of the catheter tip.

As used herein, the term “dilator” refers to a long, tapered device adapted stretch an opening in skin and/or to stretch a blood vessel to allow for insertion of a larger device, e.g., a sheath, catheter, etc.

The term “distal”, as used herein, refers to the state of being situated away from the interface or entry point of the device of the current invention and the patient.

The term “deflection”, as used herein, refers to movement of a catheter tip independent of the rest of the catheter.

The term “dyscrasia”, as used herein, refers to an abnormal or disordered state of the body or a bodily part. The term “blood dyscrasia”, as used herein, refers to an abnormally of blood cells or of clotting elements.

The term “embolus” (plural “emboli”), as used herein, refers to a gaseous or particulate matter that acts as a traveling “clot”. A common example of an embolus is a platelet aggregate dislodged from an atherosclerotic lesion. The dislodged platelet aggregate is transported by the bloodstream through the cerebrovasculature until it reaches a vessel too small for further propagation. The dot remains there, clogging the vessel and preventing blood flow from entering the distal vasculature. Emboli can originate from distant sources such as the heart, lungs, and peripheral circulation, which could eventually travel within the cerebral blood vessels, obstructing flow and causing stroke. Other sources of emboli include atrial fibrillation and valvular disease. The severity of stroke depends on the size of the embolus and the location of the obstruction. The bigger the embolus and the larger the vessel obstruction, the larger the territory of brain at risk (Hademenos G. J. and Massoud T. F. Stroke 1997; 28:2067-2077).

The term “endoluminal”, as used herein, refers to the state of being within a tubular organ or structure (e.g., blood vessel, duct, gastrointestinal tract, etc.) or within a lumen. The term “lumen”, as used herein, refers to the inner open space or cavity of a tubular structure.

The term “French” (abbreviated “Fr” or “F” or “Fg” or “Ga” or “CH” or “Ch”), as used herein, is a system used to measure the diameter of a catheter. The French unit of measure is equivalent to three times the diameter in millimeters (mm). For example, 9 Fr is equivalent to a diameter of 3 mm.

The term “grips” as used herein refers to a part or attachment of the endovascular device by which the device is held in the hand.

The term “hemorrhage”, as used herein, refers to the escape of blood from a ruptured blood vessel.

Blood vessels are typically structurally adept to withstand the dynamic quantities required to maintain circulatory function. For reasons that are not entirely understood, the vessel wall can become fatigued and abnormally weak and possibly rupture. With vessel rupture, hemorrhage occurs with blood seeping into the surrounding brain tissue. As the blood accumulates within the brain, the displaced volume causes the blood, now thrombosed, to ultimately compress the surrounding vessels. The compression of vessels translates into a reduced vessel diameter and a corresponding reduction in flow to surrounding tissue, thereby enlarging the insult (See, e.g., Hademenos G. J. and Massoud T. F. Stroke 1997; 28:2067-2077).

In the brain, hemorrhage may occur at the brain surface (extraparenchymal), for example, from the rupture of congenital aneurysms at the circle of Willis, causing subarachnoid hemorrhage (SAH). Hemorrhage also may be intraparenchymal, for example, from rupture of vessels damaged by long-standing hypertension, and may cause a blood clot (intracerebral hematoma) within the cerebral hemispheres, in the brain stem, or in the cerebellum. Hemorrhage may be accompanied by ischemia or infarction. The mass effect of an intracerebral hematoma may compromise the blood supply of adjacent brain tissue; or SAH may cause reactive vasospasm of cerebral surface vessels, leading to further ischemic brain damage. Infarcted tissue may also become secondarily hemorrhagic. Among the vascular lesions that can lead to hemorrhagic strokes are aneurysms and arteriovenous malformations (AVMs) (See, e.g., Hademenos G. J. and Massoud T. F. Stroke 1997; 28:2067-2077).

The term “hypoplasia”, as used herein, refers to a condition of arrested development in which an organ or other part of the body remains below the normal size or in an immature state, usually due to a deficiency in the number of cells; atrophy due to destruction of some of the elements and not merely to their general reduction in size.

The term “introducer”, as used herein, refers to an instrument such as a tube or a sheath that is placed within a vein or artery for introduction of a flexible device, for example, a catheter, needle, wire, etc.

The terms “ischemic” and “ischemia”, as used herein, refer to deficient supply of blood to a body part generally due to obstruction of the inflow of arterial blood (e.g., by the narrowing of arteries, spasm or disease).

The term “kickback”, as used herein refers to the phenomenon of catheter coil prolapse (slipping forward or down) due to a counterforce against the catheter by the prolapsed coil tail. The counterforce may be due to a lack of available space to insert the last coil. This lack of space may be the result of, for example, a blood vessel variation such as a bovine arch variation, a vertebral artery variation, a thrombus, an embolus, an arteriovenous malformation and the like.

The term “myocardial infarction”, as used herein, refers to death of cells of an area of heart muscle as a result of oxygen deprivation, which in turn is caused by obstruction of the blood supply; commonly referred to as a “heart attack”. The most common cause is thrombosis of an atherosclerotic coronary artery or a spasm. Less common causes include coronary artery abnormalities and vasculitis (inflammation of blood vessels).

The term “recanalization”, as used herein, refers to the process of restoring flow to or reuniting an interrupted channel of a bodily tube (e.g., a blood vessel).

The term “reperfusion”, as used herein, refers to restoration of the flow of blood to a previously ischemic organ or tissue (e.g., heart or brain).

The term “restenosis”, as used herein, refers to the recurrence of abnormal narrowing of a blood vessel (e.g., artery or vein) or valve.

The term “steerability”, as used herein, refers to an ability to turn or rotate the distal end of a catheter with like-for-like movement of the proximal section or the catheter handle.

The term “stroke” or “cerebrovascular accident”, as used herein, refers to neurological signs and symptoms, usually focal and acute, which result from diseases involving blood vessels. Strokes are either occlusive (due to closure of a blood vessel) or hemorrhagic (due to bleeding from a vessel). Although most occlusive strokes are due to atherosclerosis and thrombosis, and most hemorrhagic strokes are associated with hypertension or aneurysms, strokes of either type may occur at any age from many causes, including cardiac disease, trauma, infection, neoplasm, blood dyscrasia, vascular malformation, immunological disorder, and exogenous toxins. An ischemia stroke results from a lack of blood supply and oxygen to the brain that occurs when reduced perfusion pressure distal to an abnormal narrowing (stenosis) of a blood vessel is not compensated by autoregulatory dilation of the resistance vessels. When ischemia is sufficiently severe and prolonged, neurons and other cellular elements die. This condition is referred to as “infarction” (See, e.g., Hart R. G. et al., Stroke 1990; 21:1111-1121). Although the consequences of both ischemic and hemorrhagic stroke are similar (i.e., vessel obstruction, resultant reduced blood flow to the brain, neurological deficits and possibly death), the biophysical and hemodynamic mechanisms behind the obstruction of blood flow are different. Biophysical mechanisms for the development of obstructions that ultimately lead to stroke can arise by six distinct processes: atherosclerosis, embolus, thrombus, reduced systemic pressure, hemorrhage, and vasospasm (See, e.g., Hademenos G. J. and Massoud T. F., Stroke 1997; 28:2067-2077).

As used herein, the term “taper” refers to a reduction of thickness toward one end; the gradual diminution of width or thickness in an elongated object, i.e. to become more slender toward one end.

The term “thrombectomy”, as used herein, refers to the surgical excision of a thrombus.

The term “thrombus”, as used herein, refers to an internal physiological mechanism responsible for the clotting of blood. A thrombus is a blood clot, an aggregation of platelets and fibrin formed in response either to an atherosclerotic lesion or to vessel injury. In response to vessel or tissue injury, the blood coagulation system is activated, which initiates a cascade of processes, transforming prothrombin, ultimately resulting in a fibrin clot (See, e.g., Hademenos G. J. and Massoud T. F. Stroke 1997; 28:2067-2077).

Although a host of mechanisms and causes are responsible for vessel injury, vessel injury can occur as a result of forces (e.g., shear stresses) coupled with excess energy created by the turbulent flow exerted against the inner (intimal) lining of the vessel wall, particularly an atherosclerotic vessel wall (See, e.g., Fry D. L. Circ Res. 1968; 22:165-197; Stein P. D. and Sabbah H. N. Circ Res. 1974; 35:608-614; Mustard J. F. et al. Am J Med. 1962; 33:621-647; Goldsmith H. L. et al. Thromb Haemost 1986; 55:415-435).

The term “tortuosity” and other grammatical forms of the term “tortuous” is used herein to refer to a property of a tube, passage or blood vessel (e.g., an artery or a vein) being twisted, crooked or having many turns.

The term “vasospasm”, as used herein, refers to the sudden constriction of a blood vessel, reducing its diameter and flow rate. When bleeding occurs in the subarachnoid space, the arteries in the subarachnoid space can become spastic with a muscular contraction, known as cerebral vasospasm. The contraction from vasospasm can produce a focal constriction of sufficient severity to cause total occlusion. The length of time that the vessel is contracted during vasospasm varies from hours to days. However, regardless of the duration of vessel constriction, reduction of blood flow induces cerebral ischemia, thought to be reversible within the first 6 hours and irreversible thereafter. It has been shown that vasospasm is maximal between 5 and 10 days after subarachnoid hemorrhage and can occur up to 2 weeks after subarachnoid hemorrhage (See, e.g., Wilkins R. H. Contemp Neurosurg. 1988; 10:1-66; Hademenos G. J. and Massoud T. F. Stroke 1997; 28:2067-2077).

In the various views of the drawings, like reference characters designate like or similar parts.

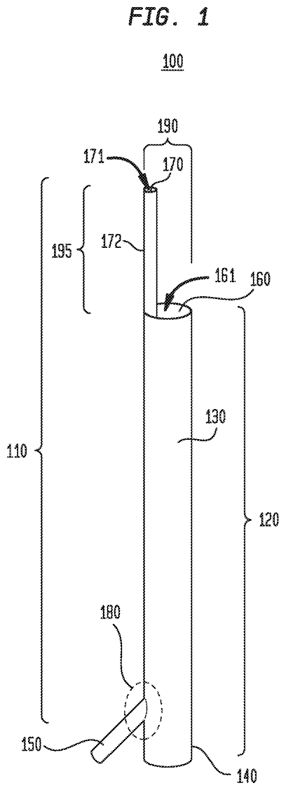

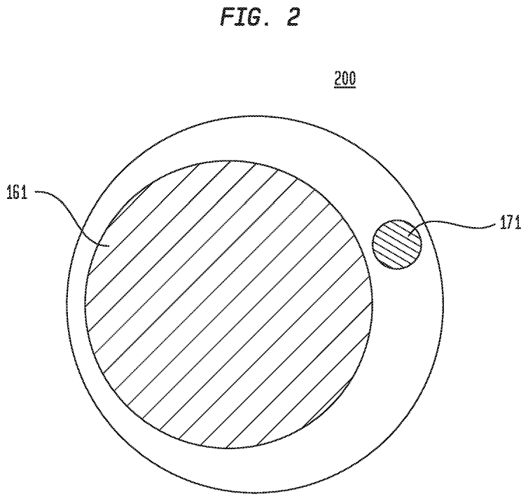

FIGS. 1 and 2 show an exemplary and non-limiting example of some embodiments of the endovascular device 100 of the invention. According to one configuration, FIG. 1 illustrates a side view of the endovascular device 100 comprising a tube 130 defining a working lumen 161. According to some embodiments, tube 130 further comprises a support tube 172 defining a support lumen 171. According to some embodiments, tube 130 comprises a bifurcation 180 at a first end and openings 190 at a second end. The bifurcation comprises a first branch 140 and a second branch 150. The openings comprise a primary opening 160 leading to the working lumen 161 and a secondary opening 170 leading to the support lumen 171. The first branch 140, the working lumen 161, and the primary opening 160 form a working segment 120, which contains both the entirety of the working lumen 161 and the proximal portion of the support lumen 171. A segment 110 incorporates the entirety of the device. The part of the support tube 172 distal to the primary opening 160 comprises the distal support segment 195, which incorporates the distal segment of the support lumen 171 and the secondary opening 170. According to some embodiments, a luer lock is attached to the proximal end (first end) of each branch. FIG. 2 illustrates a cross-sectional view 200 through segment 120 of the endovascular device 100 comprising a cross-sectional view of the working lumen 161 and a cross-sectional view of the support lumen 171 at the middle of working segment 120 of the device.

According to some embodiments, the distal support segment 195 and or the working segment 120 is rigid. According to some embodiments, the distal support segment 195 and/or the working segment 120 comprises a soft, flexible portion.

According to some embodiments, the endovascular device 100 is an intracranial endovascular device. According to some embodiments, the endovascular device is a peripheral blood vessel endovascular device. According to some embodiments, the endovascular device is a cardiac blood vessel endovascular device.

Preferred embodiments of the present invention do not use pre-formed, curved configurations to keep the catheter in place. Different pre-formed curved configurations are required for different target areas. Not using pre-formed curved configuration facilitates positioning, and increases flexibility of use due to the ability to use the same configuration for different target areas.

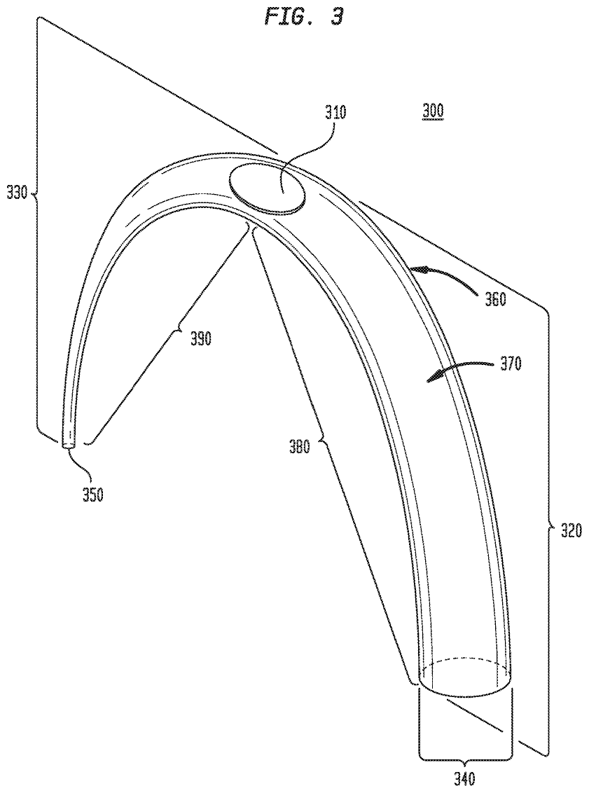

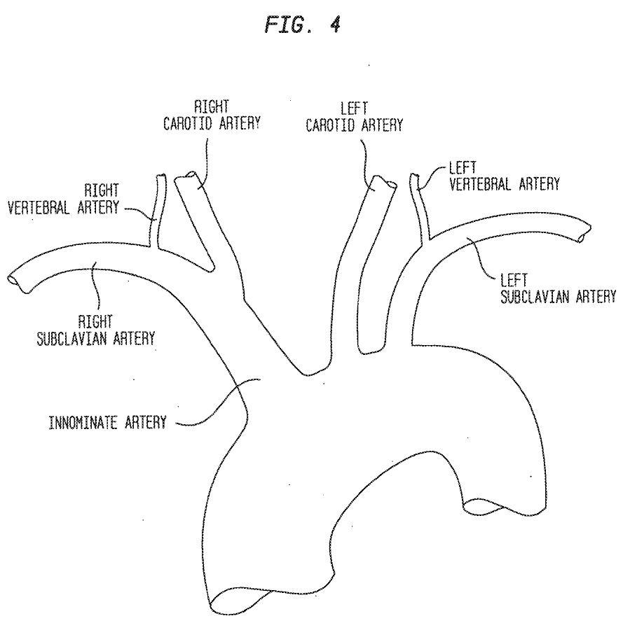

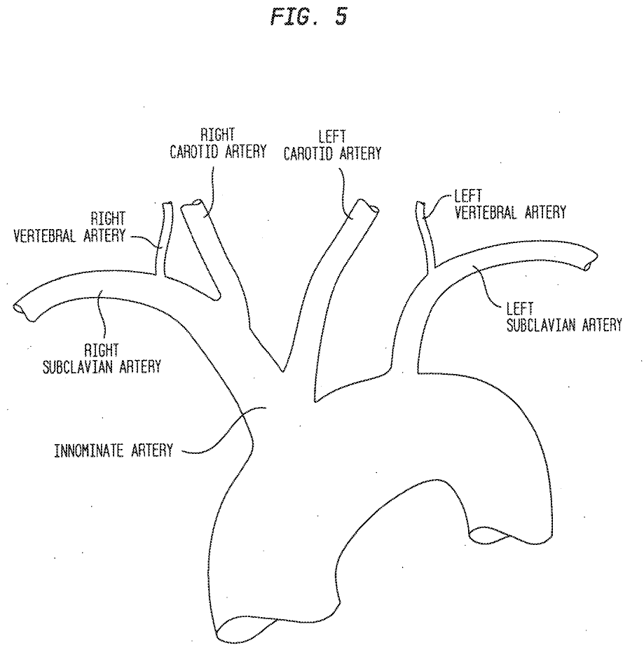

FIG. 3 shows an exemplary and non-limiting example of some embodiments of the endovascular device 300 in side view. Endovascular device 300 comprising a tube 360 defining a lumen 370, a side-hole 310, a primary opening 340, and an open end 350. The side-hole 310 divides the endovascular device 300 into two (2) segments: a first segment 320 and a second segment 330. The first segment 320 comprises primary opening 340. First segment 320 extends from primary opening 340 to side-hole 310. First segment 320 forms a working segment 380 comprising primary opening 340, lumen 370, and side-hole 310. The second segment 330 extends from the side-hole 310 and can taper (i.e., decrease in diameter) to an end 350. In some embodiments, it need not taper. The second segment 330 forms support segment 390 comprising end 350 and lumen 370.

According to some embodiments, at least part of first segment 320 and/or second segment 330 of the endovascular device depicted in FIG. 3 is preformed in the form of a geometric shape as described herein.

According to some embodiments, the second segment 330 extends from the side-hole 310 and tapers externally to an end hole 350 and maintains a constant inner lumen diameter that does not substantially decrease at a distal end. According to these embodiments, a second segment 330 inner lumen with a diameter that does not substantially decrease enhances the anchoring properties of the second segment 330, which will press more readily against the blood vessel walls at bends in the blood vessel.

According to some embodiments, the second segment 330 has an inner lumen that is circular in cross section. According to some embodiments, the second segment 330 has an inner lumen that is oval in cross section. According to some embodiments, the second segment 330 has an inner lumen that is flattened in cross section. According to some embodiments, the second segment 330 maintains a constant inner lumen diameter. According to some embodiments, the second segment 330 has an inner lumen diameter that varies along the course of the endovascular device.

According to some embodiments, the length of the working segment 380 formed between the side-hole 310 and the first segment 320 of the endovascular device ranges from about 20 cm to about 160 cm. According to some embodiments, the inner diameter (ID) of the lumen 370 in the working segment of the endovascular device ranges from about 0.1 French (Fr) (0.001 inches) to about 30 French (Fr) (0.394 inches). According to some embodiments, the diameter of the lumen 370 of first segment 320 is less than 1 French (Fr). According to some embodiments, the diameter of the lumen 370 of the second segment 330 ranges from about 0.1 French (Fr) to at least 30 French (Fr).

According to some embodiments, the length of support segment 390 of endovascular device 300 for intracranial applications ranges from about 0.01% to about 20% of the length of the working segment 380. For example, according to some embodiments, the length of the support segment 390 of the endovascular device for intracerebral applications ranges from about 0.05 cm to about 32 cm.

According to some embodiments, the length of the support segment 390 of the endovascular device for peripheral applications ranges from about 0.01% to about 200% of the length of the working segment 380. For example, according to some embodiments, the length of the support segment 390 of the endovascular device for peripheral applications ranges from about 0.05 cm to about 320 cm. According to some embodiments, the second segment 330 extends from about 0.6 cm to about 200 cm in length from the side-hole 310.

According to some embodiments, the end 350 is open. According to some embodiments, the end 350 is closed.

According to some embodiments, endovascular device 300 is the inner catheter of endovascular device 1400 described below.

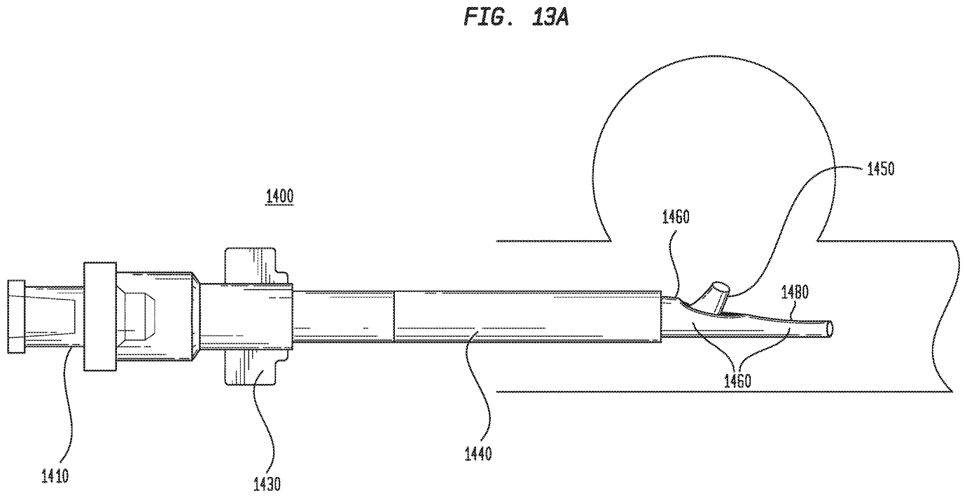

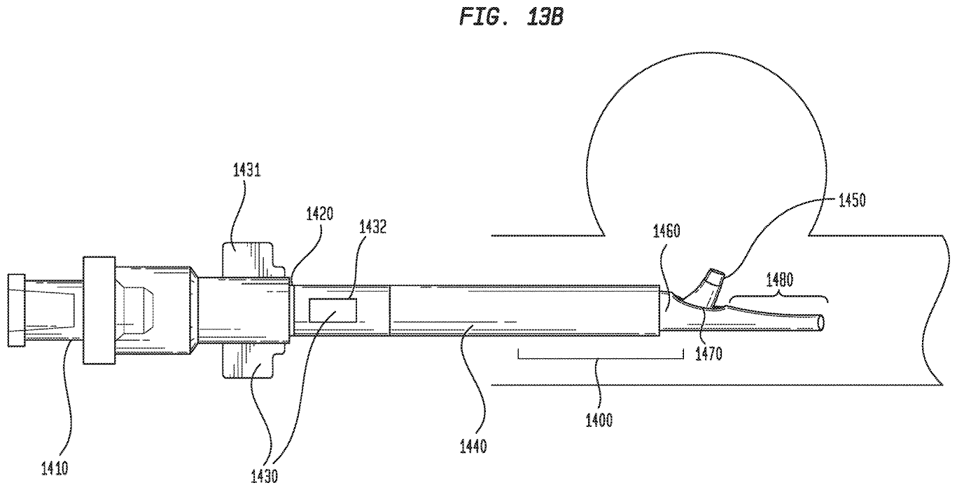

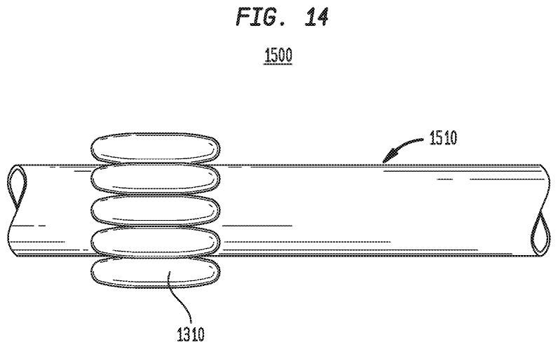

According to some embodiments, endovascular device 1400 comprises an outer support catheter 1440 and an inner catheter 1460 disposed at least partially within the lumen defined by an outer support catheter 1440 (See, e.g., FIG. 13). According to some embodiments, the inner catheter 1460 comprises a side-hole. According to some embodiments, the side-hole is a working distal side-hole 1470. According to some embodiments, the working distal side-hole comprises an angled extension 1450. According to some embodiments, a distal support segment 1480 (corresponding to distal support segment 330 of FIG. 3) that extends beyond the distal working side-hole 1470 (corresponding to working side-hole 310 of FIG. 3), serves to provide additional support and stability to any devices being advanced through catheter 1460 and through side hole 1470 into a lesion or an acutely angled and/or tortuous vessel, and minimizes the tendency of such device and/or its delivery wire to “kick-back” into the parent vessel.

According to some embodiments, the support segment can be used to anchor/support a wire and/or microcatheter to access a branch vessel arising at a difficult angle. This is especially common with branches feeding AVM's. Using conventional techniques, the wire and/or catheter will herniate out of the branch and into the distal parent vessel as it is advanced, especially when additional bends/curves of the branch vessel are encountered.

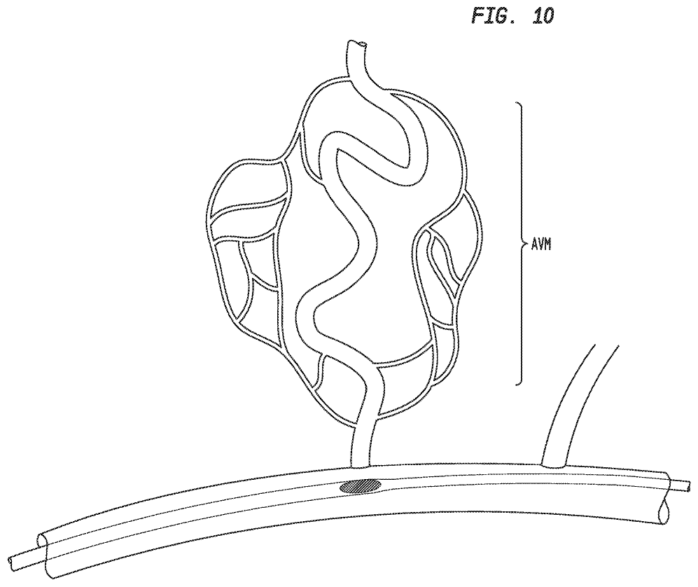

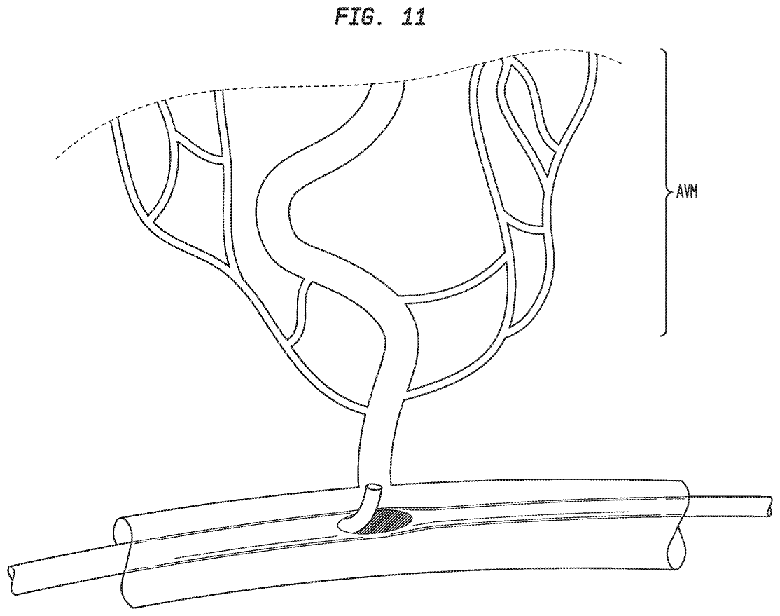

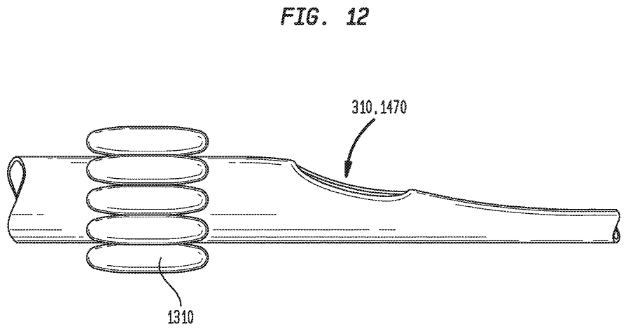

FIGS. 10 and 11 show an endovascular device, according to some embodiments. The device is depicted inserted in the left middle cerebral artery (L-MCA) with the side-hole oriented to access an acutely angled branch feeder of an arteriovenous malformation (AVM). FIG. 11 shows a catheter being advanced out of the side-hole and into the branch feeder of the AVM.

According to some embodiments, one or more radiopaque markers can be used to identify the position of the working distal side-hole. According to some embodiments, intravascular ultrasound (IVUS) can be incorporated into the catheter to help identify the position of the working side-hole relative to a lesion such as an aneurysm, a vessel or other targeted anatomy.

According to some embodiments, grips are connected to the inner and outer catheters. According to some such embodiments, inner catheter 1460 is connected to grips on the inner catheter 1431 at the proximal end of the endovascular device (FIG. 13A). A separate set of grips 1432 is on the proximal end of the outer catheter 1440. According to some embodiments, the operator uses the grips to rotate the inner catheter 1460 relative to the outer catheter 1440. The operator can use the grips 1432 on proximal end of the outer catheter 1440 to hold the outer catheter in a stable position, while using the grips 1431 on the inner catheter 1460 relative to the fixed outer catheter and thereby reposition the working side-hole 1470 in a different desired orientation. According to some embodiments, the rotation orients the distal working side-hole 1470 of device 1400 in a desired direction and location. According to some embodiments, endovascular device 1400 can be rotated within a blood vessel. According to some embodiments, the rotation of endovascular device 1400 is effective to position inner catheter 1460 within a blood vessel. According to some embodiments, the rotation is effective to position the working side-hole of inner catheter 1460 within a blood vessel. According to some embodiments, the rotation is effective to center the working distal side-hole of inner catheter 1460 on the base of an aneurysm. According to some embodiments, the rotation is effective to center the working distal side-hole of inner catheter 1460 in proper alignment with a blood vessel origin and for support in accessing the blood vessel.

According to some embodiments, endovascular device 1400 comprises one or two sets of grips 1430 at the first (proximal) end of the device (FIG. 13B). According to some embodiments, the grips are used by the operator to rotate the inner or outer catheter of the endovascular device 1400.

According to some embodiments, a first set of grips can be connected to inner catheter 1460 and a second set of grips can be connected to outer catheter 1440.

By way of example, the grips 1430 of outer support catheter 1440 can be immobilized/held stationary while the grips of inner catheter 1460 are rotated relative to the grips of the outer support catheter. Put another way, the outer support catheter is maintained in a fixed position while rotating the inner catheter relative to the outer support catheter.

According to some embodiments, inner catheter 1460 of endovascular device 1400 is endovascular device 100 or endovascular device 300. While endovascular device 100 would allow for more support, it would be bulkier and may be more difficult to rotate than endovascular device 300.

It should be understood that the inner/outer catheter arrangement with grips as described above and shown in FIGS. 13A and 13B is only one illustrative way to achieve rotation. According to some embodiments, for example, a single balloon on the inner catheter just beside the working side-hole is effective to deflect the catheter away from an aneurysm and provide more working room; a circumferential array of balloons can achieve the same purpose, with more variations of deflection capabilities. According to some embodiments, a symmetric or asymmetric balloon arranged so that it can deflect the catheter away from a base/neck of the aneurysm while also occluding a vessel, thereby allowing flow arrest and/or flow reversal and decreasing the tendency for normal flow to deflect the advancing endovascular device. In some embodiments, no balloon is on the inner catheter. In some embodiments, one or more balloons are on the outer catheter. In some embodiments, no balloons are on the outer catheter.

Many catheters that are flexible enough to deliver devices intracranially are not stiff enough for proximal rotation at the catheter hub outside the body to result in the intracranial portion rotating-rather, the shaft of the catheter shaft will twist into an unusable spiral.

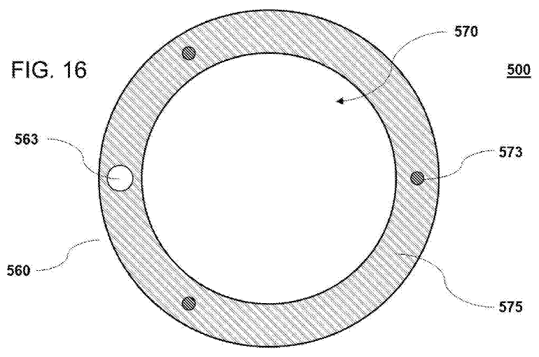

One approach to solve this limitation is to provide spiral “pulley” wires within the catheter wall that are attached proximally to a wheel or similar device (without the spiral this approach is used to actively bend catheters at certain inflection points). When the wires are shortened, the wire spiral straightens/unwinds, and the catheter tip will rotate into the desired orientation. FIG. 16 illustrates a cross-sectional view 500 through segment 560 of the endovascular device, according so some embodiments, comprising a cross-sectional view of the working lumen 570 and a cross-sectional view of three spiraling pulley wires 573 substantially within the wall 575 of the intravascular portion of the device. In addition, there is also a smaller additional lumen 563 substantially within the wall 575 of the intravascular portion of the device that serves as a support lumen through which an additional support wire or support balloon or other endovascular device. According to some embodiments, the support lumen 563 serves solely to inflate and deflate the at least one balloon. The support lumen 563 also spirals in this configuration to avoid support lumen 563 crossing with pulley wires 573.

Another approach is for the endovascular device to have multiple side-holes, either circumferentially at the same length along the catheter, or staggered slightly, or some combination thereof. The appropriate hole can then be selected with the wire and/or catheter being used to access the side branch or aneurysm. Preferable, each side-hole should not have a side-hole directly across from it, in order to maximize the support for the devices exiting that side-hole. Side holes can optionally have various radiographic markers at various junctions along their periphery to help identify their location during a procedure.

According to some embodiments, a circumferential array of balloons is attached to the outside of the outer catheter 1440. According to some embodiments, the balloons are attached to the outside of the distal, exposed segment of the inner catheter 1460. According to some embodiments, the circumferential balloons can be on the outer sheeth of the catheter. According to some embodiments, the balloons are attached to both the outside of the outer catheter 1440 and the outside of the distal, exposed segment of the inner catheter 1460. According to some embodiments, inner catheter 1460 comprises one or more grooves. According to some embodiments, the one or more grooves are located around the outer circumference of inner catheter 1460. According to some embodiments, outer catheter 1440 comprises one or more grooves. According to some embodiments, the one or more grooves are located around the inner circumference of outer catheter 1440. According to some embodiments, the grooves allow the inner and outer catheters to rotate relative to each other while preventing movement along the length of one catheter relative to the other catheter. By way of example, the inner catheter can comprise an outer groove along the outside of the inner catheter, centered between two (2) inner grooves on the inner surface of the outer catheter at a corresponding location. According to some embodiments, there can be an inner groove along the inside of the outer catheter, centered between two (2) outer grooves on the outside of the inner catheter. According to some embodiments, the inner and outer catheters may comprise additional grooves for added translational stability.

By inflating some balloons and not others, the catheter can be centered at the desired location within a vessel. According to some such embodiments, a catheter can be centered at the base of a bifurcation aneurysm, for subsequent delivery of a WEB® device, coils or another device. According to some such embodiments, the balloons can be on an intermediate support catheter, or directly on the delivering microcatheter.

According to some embodiments, the aneurysm treatment device comprises a WEB® device (Microventions Inc., CA). The WEB® device is most safely deployed via a catheter centered within the aneurysm. Using conventional technologies, this is most often most safely possible with bifurcation aneurysms. However, it is very difficult to center a catheter in most side-wall aneurysms. The catheter will typically rather sit against the distal side wall of the aneurysm. Even when possible, it is typically with poor support, and the catheter may kick out during device advancement. The endovascular device, according to some embodiments, can provide the stability and support to appropriately position a catheter, safely and with adequate support, to deliver a WEB® and similar devices into most side wall aneurysms.

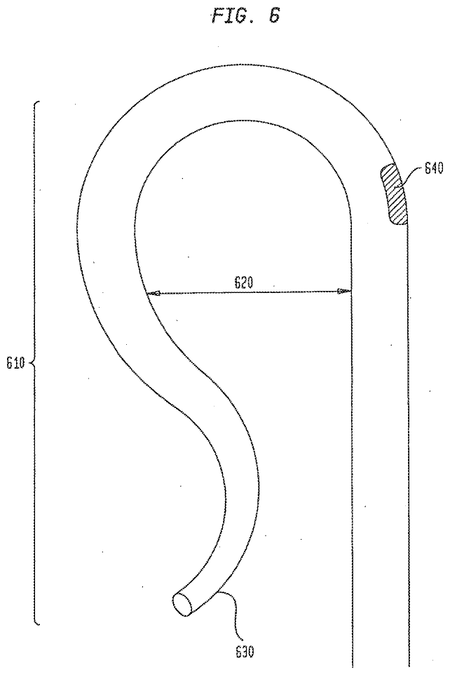

According to some embodiments, the shape of support lumen 110 or support lumen 330 is an “S” shape. According to some embodiments, the “S” shape is a shepherd's hook shape. FIG. 6 shows an exemplary and non-limiting example of one embodiment of the “S”-shaped support lumen 610.

According to some embodiments, the “S”-shaped support lumen 610 can be used to access difficult-to-access innominate arteries; to subsequently access the right common carotid artery and its distal branches; and/or to subsequently access the right subclavian artery and/or its branches. By way of non-limiting example, in a subject with an overgrown, tortuous and/or long aortic arch, and/or an elongated, straightened and/or tortuous innominate artery, the “S”-shaped support lumen 610 can be used to distally access the right subclavian artery and right common carotid artery.

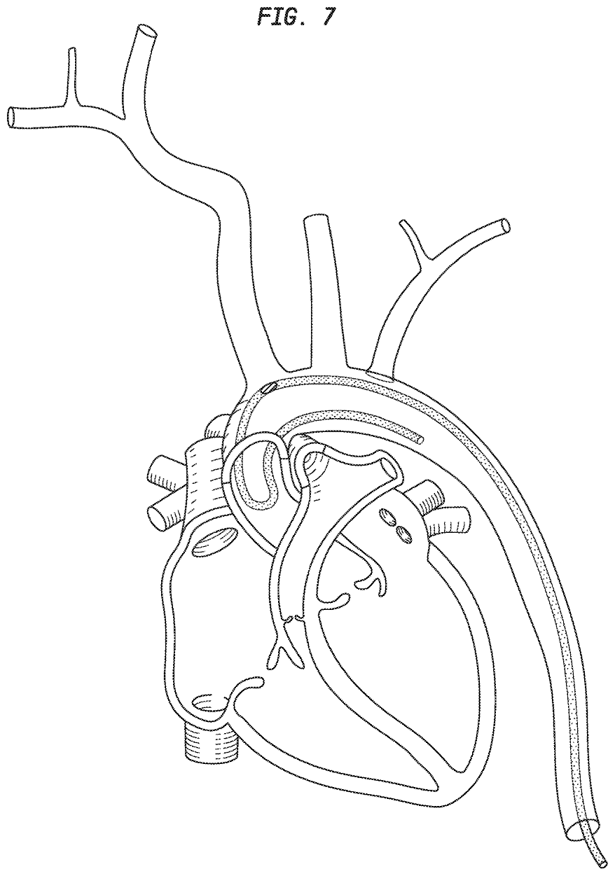

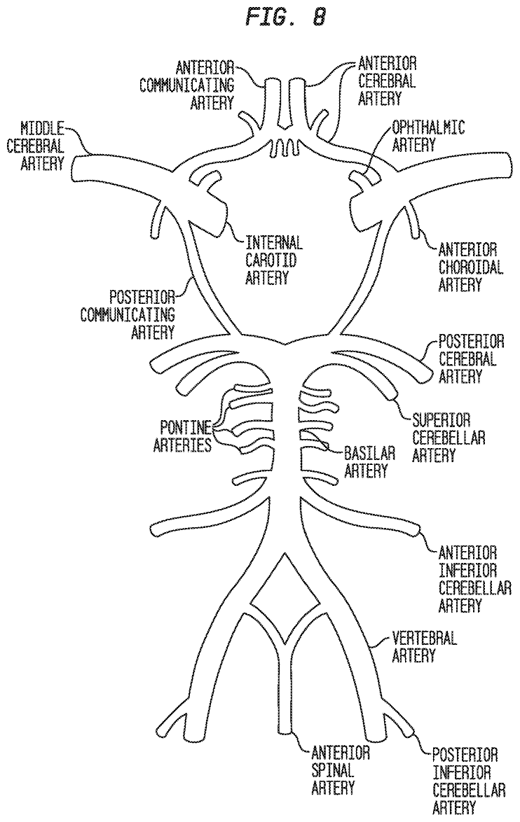

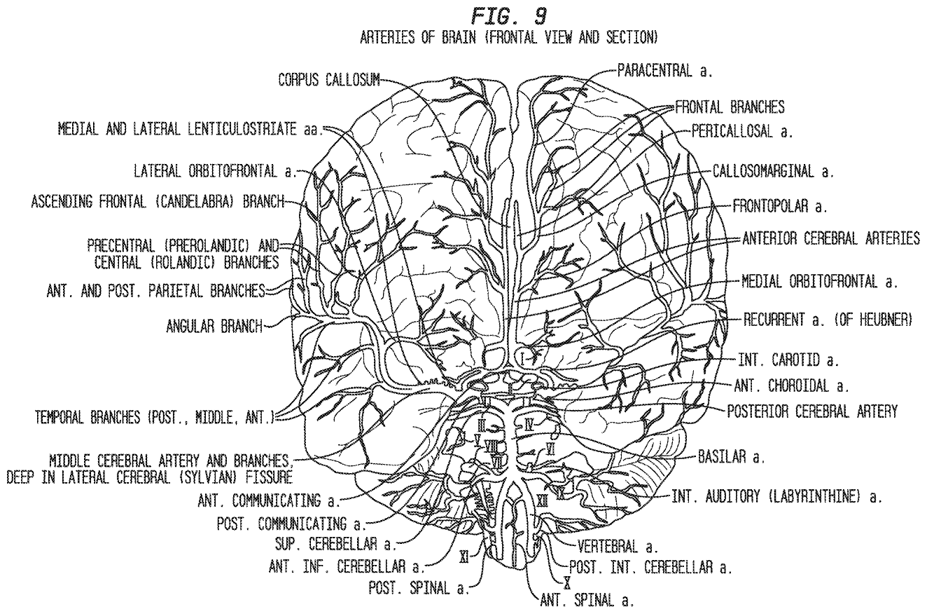

According to some embodiments, the “S” shape is a pre-shaped configuration. According to some embodiments, the “S”-shaped support lumen 610 is inserted into the body of a subject in a straight configuration and subsequently re-shaped into its pre-shaped “S” configuration. According to some embodiments, the “S”-shaped support lumen 610 is inserted into the body of a subject in a straight configuration, into the aortic arch in a straight configuration, and the distal portion 630 of the “S”-shaped support lumen 610 curves back across the aortic arch to provide support for the endovascular device 300, to facilitate placement of the endovascular device 300, to anchor the endovascular device 300 within a blood vessel, to prevent kickback of the endovascular device 300, or a combination thereof (FIG. 7).

According to some embodiments, the “S”-shaped support lumen 610 is inserted into the body of a subject in a straight configuration, into the aortic arch in a straight configuration, and the distal portion 630 of the “S”-shaped support lumen 610 curves back across the aortic arch and down the descending aorta to provide support for the endovascular device 300, to facilitate placement of the endovascular device 300, to anchor the endovascular device 300 within a blood vessel, to prevent kickback of the endovascular device 300, or a combination thereof (FIG. 7). According to some embodiments, the placement of the endovascular device 300 is with the side-hole 310/640 positioned at the origin of the innominate artery (FIG. 7).

According to some embodiments, the “S”-shaped support lumen 610 comprises a shape memory polymer (SMP). Shape memory polymers include, but are not limited to methacrylates, polyurethanes, blends of polystyrene and polyurethane, and polyvinylchloride. According to some embodiments, the “S”-shaped support lumen 610 comprises a shape memory alloy (SMA). Non-limiting examples of shape memory alloys include nickel-titanium (i.e., nitinol).

According to some embodiments, the length of the “S”-shaped support lumen 610 is about 4% to about 300% longer than the length of the working lumen 120. According to some embodiments, the “S”-shaped support lumen 610 comprises a curve diameter 620. According to some embodiments, the curve diameter ranges from about 1 cm to about 10 cm. According to some embodiments, the curve diameter ranges from about 2 cm to about 8 cm.

According to some embodiments, no ramp is disposed within said tube at or near said side-hole. The absence of such a ramp results in increased flexibility of the endovascular device relative to an endovascular device with such a ramp (See, e.g., U.S. Pat. No. 4,552,554). In addition, the presence of such a ramp as found in some endovascular devices results in unnecessary upsizing of the outer tube, which leads to more difficult navigation and increased risks of access site complications due to the larger hole in the device. The presence of such a ramp also limits what can be placed into the distal portion of the device to smaller wires, while forcing larger wires to exit the side port.

According to some embodiments, the primary opening 340 comprises a luer lock. According to some embodiments, the length of the first segment 320 from the luer lock to the side-hole 310 ranges from about 10 cm to about 130 cm. According to some embodiments, the length of the second segment 330 ranges from about 10% to about 300% longer than the length of the first segment 320 from the luer lock to the side-hole 310.

According to some embodiments, the length of the endovascular device 300 from the luer lock to the end 350 ranges from about 10 cm to about 520 cm.

According to some embodiments, the second segment 330 provides stability to the endovascular device 300 and/or the working lumen formed by the side-hole 310 and the first segment 320. According to some embodiments, the second segment 330 provides strength to the endovascular device 300 and/or the working lumen. According to some embodiments, the second segment 330 provides support for the endovascular device 300 and/or the working lumen. According to some embodiments, the second segment 330 facilitates placement of the endovascular device 300 and/or the working lumen. According to some embodiments, the second segment 330 anchors the endovascular device 300 and/or the working lumen within a blood vessel. According to some embodiments, the second segment 330 prevents kickback of the endovascular device 300 and/or the working lumen. According to some embodiments, the blood vessel is an artery. According to some embodiments, the blood vessel is a vein. According to some embodiments, the device can be used in other lumens.

According to some multilumen embodiments, the length of the support lumen 110 is greater than the length of the working lumen 120. According to some embodiments, the length of the support lumen 110 ranges from about 0.1% to about 200% longer than the working lumen 120. According to some embodiments, the length of the support lumen 110 ranges from about 105 cm to about 135 cm. According to some embodiments, the length of the working lumen 120 ranges from about 60 cm to about 90 cm.

According to some embodiments, the length of the first segment 320 ranges from about 50 cm to about 100 cm. According to some embodiments, the second segment 330 extends from about 20 cm to about 60 cm in length from the side-hole 310.

According to some “single lumen” (exclusive of any additional lumens for wires or balloon, which most often would be substantially within the wall of the catheter) embodiments the length of the intravascular portion of the first segment ranges from 5 cm to 150 cm. According to some embodiments, the length of the second segment ranges from 0.1 cm to 120 cm of the second segment According to some embodiments, the length of the second segment is between 0.1%-300% the length of the first segment. According to some multilumen embodiments, the support lumen spans the entire length of the working lumen and extends beyond the side hole as well to form the support segment. According to some “single lumen” embodiments the support lumen begins only after the side hole, and is contained only within the support segment, and does not span the length of the working lumen.

According to some embodiments, the outer diameter of the working segment is the same as the outer diameter of the support segment. According to some embodiments, some or all of the diameter of the support segment is less than the outer diameter of the working segment.

According to some embodiments, the diameter of the support lumen 110 is less than the diameter of the working lumen 120. According to some embodiments, the diameter of the support lumen 110 ranges from about 1 French (Fr) to about 8 French (Fr). According to some embodiments, the diameter of the support lumen 110 is less than 1 French (Fr). According to some embodiments, the diameter of the support lumen 110 ranges from about 0.0020 cm (about 0.0008 inches) to about 0.1 cm (about 0.039 inches). According to some embodiments, the diameter of the support lumen 110 ranges from about 0.08 cm to about 1 cm.

According to some embodiments, the diameter of the working lumen 120 ranges from about 1 French (Fr) to about 26 French (Fr). According to some embodiments, the diameter of the working lumen 120 is less than 1 French (Fr). According to some embodiments, the diameter of the working lumen 120 ranges from about 0.0254 cm (about 0.010 inches) to about 0.1 cm (about 0.039 inches). According to some embodiments, the diameter of the working lumen 120 ranges from about 0.1 cm to about 1 cm.

According to some embodiments, the first segment 320 ranges in diameter from about 1 French (Fr) to about 26 French (Fr). According to some embodiments, the diameter of the first segment 320 is less than 1 French (Fr). According to some embodiments, the diameter of the first segment 320 ranges from about 0.0254 cm (about 0.010 inches) to about 0.0305 cm (about 0.012 inches).

According to some embodiments, the diameter of the second segment 330 ranges from about 1 French (Fr) to about 23 French (Fr). According to some embodiments, the diameter of the second segment 330 is less than 1 French (Fr). According to some embodiments, the diameter of the second segment 330 ranges from about 0.0020 cm (about 0.0008 inches) to about 0.1 cm (about 0.039 inches. According to some embodiments, the diameter of the second segment 330 ranges from about 0.1 cm to about 1 cm.

According to some embodiments, the diameter is an inner diameter (ID). According to some embodiments, the diameter is an outer diameter (OD).

According to some embodiments, the support lumen 110 further comprises a device separate from the endovascular device 100. According to some embodiments, the separate device is a wire. According to some embodiments, the wire is capable of being advanced into a blood vessel through the support lumen 110. According to some embodiments, the wire provides stability to the endovascular device 100. According to some embodiments, the wire provides strength to the endovascular device 100. According to some embodiments, the wire provides support for the endovascular device 100. According to some embodiments, the wire facilitates placement of the endovascular device 100. According to some embodiments, the wire anchors the endovascular device 100 within a blood vessel. According to some embodiments, the wire provides stability to the working lumen 120. According to some embodiments, the wire provides strength to the working lumen 120. According to some embodiments, the wire provides support for the working lumen 120. According to some embodiments, the wire facilitates placement of the working lumen 120. According to some embodiments, the wire anchors the working lumen 120 within a blood vessel. According to some embodiments, the blood vessel is an artery. According to some embodiments, the blood vessel is a vein.

According to some embodiments, the endovascular device 300 further comprises an additional separate device that traverses through endovascular device 300. According to some embodiments, the separate device is a wire. According to some embodiments, the wire is capable of being advanced through the first segment 320 and into the second segment 330. According to some embodiments, the wire is capable of being advanced into a blood vessel through the second segment 330. According to some embodiments, the wire provides stability to the endovascular device 300. According to some embodiments, the wire provides strength to the endovascular device 300. According to some embodiments, the wire provides support for the endovascular device 300. According to some embodiments, the wire facilitates placement of the endovascular device 300. According to some embodiments, the wire anchors the endovascular device 300 within a blood vessel. According to some embodiments, the wire provides stability to the working lumen formed by the side-hole 310 and the first segment 320. According to some embodiments, the wire provides strength to the working lumen formed by the side-hole 310 and the first segment 320. According to some embodiments, the wire provides support for the working lumen formed by the side-hole 310 and the first segment 320. According to some embodiments, the wire facilitates placement of the working lumen formed by the side-hole 310 and the first segment 320. According to some embodiments, the wire anchors the working lumen formed by the side-hole 310 and the first segment 320 within a blood vessel. According to some embodiments, the blood vessel is an artery. According to some embodiments, the blood vessel is a vein. According to some embodiments, the additional device is a catheter. According to some embodiments, the additional device is a stent. According to some embodiments, the additional device is a balloon. According to some embodiments, the additional device is an embolic device. According to some embodiments, the additional device is a different device. According to some embodiments, the additional device is a combination of various devices, used simultaneously or in sequentially.

According to some embodiments, the diameter of the wire ranges from about 0.07 cm to about 0.11 cm. According to some embodiments, the wire is rigid. According to some embodiments, the wire is flexible.

According to some embodiments, the wire is comprised of a core material which includes, but is not limited to, stainless steel, nitinol or a combination thereof. In general, stainless steel is easier to torque and is more rigid, providing better columnar support. Nitinol is more flexible and kink resistant. Developments such as high-tensile-strength stainless steel and combinations of stainless steel with nitinol have been utilized. High-tensile-strength stainless steel provides more column strength and torquability than original stainless steel. The use of hybrid wires incorporates high-tensile stainless steel shafts with nitinol tips to impart high torquability and columnar shaft strength with kink-resistance tips.

According to some embodiments, the wire comprises a core taper. The core tapers are areas where the core of the wire changes over a set distance. There may be several tapers in a wire. Long, gradual tapers track well around bends, but do not provide as much support in short distances. Broad, gradual, or long tapers offer acute vessel access and improved tracking. Devices with abrupt or short tapers create support in shorter distances and have a greater tendency to prolapse.

According to some embodiments, the wire comprises a core grind (i.e., constant diameter).

According to some embodiments, the wire comprises a core that extends to the tip. A core that extends to the tip of the wire increases the transmission of force, is more durable and steerable, improves tactile feedback, and is used in peripheral vessels. According to some embodiments, the wire comprises a core that does not extend to the tip. A core that does not extend to the tip (i.e., shaping ribbon design) is delicate, flexible, and soft. This kind of tip is also easier to shape, can be easily prolapsed, and is less likely to inadvertently injure distal vessels.

According to some embodiments, the wire comprises a cover. Covers include, but are not limited to, a polymer or a plastic. A polymer sleeve or plastic placed over the wire core enhances lubricity which results in less drag, enhanced lesion crossing, and smooth tracking in tortuous vessels. According to some embodiments, the wire comprises a coating. Non-limiting examples of a coating include a hydrophobic coating and a hydrophilic coating. Hydrophobic coatings reduce friction and improve device trackability by repelling water to create a smooth, “wax-like” surface, with no water actuation required. Hydrophilic coatings attract water to create a slippery, “gel-like” surface.

According to some embodiments, the wire is a guide wire.

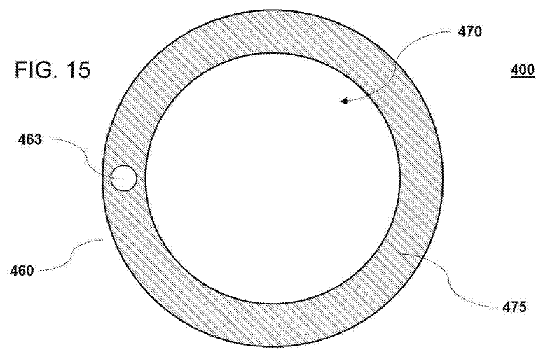

According to some embodiments, the endovascular device further comprises at least one balloon disposed thereon, and at least one additional lumen substantially within the wall (so as not to obstruct the single central working lumen) of the intravascular portion of the device that serves solely to inflate and deflate the at least one balloon. FIG. 15 illustrates a cross-sectional view 400 through segment 460 of the endovascular device, according so some embodiments, comprising a cross-sectional view of the working lumen 470 and a cross-sectional view of a smaller additional lumen 463 substantially within the wall 475 of the intravascular portion of the device that serves as a support lumen through which an additional support wire or support balloon or other endovascular device. According to some embodiments, the support lumen 463 serves solely to inflate and deflate the at least one balloon.

According to some embodiments, support segment 390 or support lumen 110 of the endovascular device comprises an inflatable balloon. According to some embodiments, the inflatable balloon is attached to the distal portion of the support segment 390 or support lumen 110 of the endovascular device. According to some embodiments, the inflatable balloon is attached proximal to the side-hole 310. According to some embodiments, the inflatable balloon is attached distal to the side-hole 310. According to some embodiments, the inflatable balloon is attached opposite the side-hole 310. According to some embodiments, the inflatable balloon is attached opposite the side-hole 310 and spans the length of the side-hole 310. According to some embodiments, the inflatable balloon is attached to a device separate from the endovascular device 300 or endovascular device 100. According to some embodiments, the separate device is a segment of an inflatable balloon catheter. According to some embodiments, the segment of the inflatable balloon catheter comprises a luer lock on its proximal end. According to some embodiments, the separate device can be advanced into a blood vessel through the lumen defined in the support segment 390 and/or the lumen defined in the working segment 380 or the support lumen 110 of the endovascular device. According to some embodiments, the inflated balloon is effective to anchor the support segment 390 or support lumen 110 of the endovascular device to a blood vessel. According to some embodiments, the blood vessel is an artery. According to some embodiments, the blood vessel is a vein.

According to some embodiments, the diameter of the inflatable balloon ranges from about 1 mm to about 50 mm. According to some embodiments, the length of the balloon ranges from about 1 mm to about 300 mm.

According to some embodiments, the inflatable balloon is comprised of various shapes including, but not limited to, cylindrical, spherical, oval, conical, stepped, tapered and dog bone.

According to some embodiments, the inflatable balloon is comprised of a material such as, for example, a polyamide, polyethylene terephthalate (PET), polyurethane, composites, and engineered nylons. Engineered nylons include, but are not limited to, PEBAX®, GRILAMID®, and VESTAMID®.

According to some embodiments, the inflatable balloon ends are comprised of various shapes including, but not limited to, conical sharp corner, conical radius corner, offset neck, spherical end and square.

According to some embodiments, the inflatable balloon is filled with a fluid. Non-limiting examples of fluid include sterile water and saline.

According to some embodiments, support lumen 110 comprises a stent. According to some embodiments, the stent is retrievable. According to some embodiments, the retrievable stent is attached to the distal portion of the support lumen 110. According to some embodiments, the retrievable stent is attached to a device separate from the endovascular device. According to some embodiments, the separate device is capable of being advanced into a blood vessel through the support lumen 110. According to some embodiments, the retrievable stent anchors the support lumen 110 to a blood vessel. According to some embodiments, the blood vessel is an artery. According to some embodiments, the blood vessel is a vein.

According to some embodiments, the stent is self-expanding. According to some embodiments, the self-expanding stent is attached to the distal portion of the support lumen 110. According to some embodiments, the self-expanding stent is attached to a device separate from the endovascular device. According to some embodiments, the separate device is capable of being advanced into a blood vessel through the support lumen 110. According to some embodiments, the self-expanding stent anchors the support lumen 110 to a blood vessel. According to some embodiments, the blood vessel is an artery. According to some embodiments, the blood vessel is a vein.

According to some embodiments, the second segment 330 or support lumen 110 is rigid. According to some embodiments, the second segment 330 or support lumen 110 comprises a soft, flexible portion. According to some embodiments, the soft, flexible portion ranges in length from about 0.1 cm to about 50 cm. According to some embodiments, the soft, flexible portion is located at the distal end of the support lumen 110 or at the end 350.

According to some embodiments, the working lumen formed by the side-hole 310 and the first segment 320 or the working lumen 120 comprises a device separate from the endovascular device. According to some embodiments, the separate device is capable of being advanced into a blood vessel through the working lumen. According to some embodiments, the separate device is capable of being advanced into a blood vessel through the side-hole 310. According to some embodiments, the blood vessel is an artery or a vein. According to some embodiments, the separate device is a diagnostic device. According to some embodiments, the separate device is a therapeutic device.

According to some embodiments, the catheter further comprises an angled extension at the side-hole. According to some embodiments, the angle of the angled extension ranges from about 10 degrees to about 180 degrees. According to some embodiments, the angled extension is soft. According to some embodiments, the angled extension is flexible. According to some embodiments, the angled extension is adjustable. According to some embodiments, the endovascular device comprises an actively adjustable angled extension extending from the side-hole serving to facilitate steering of the one or more additional devices into said blood vessel.

According to some embodiments, the angled extension comprises a shape memory polymer (SMP). Shape memory polymers include, but are not limited to methacrylates, polyurethanes, blends of polystyrene and polyurethane, and polyvinylchloride. According to some embodiments, the angled extension of the catheter comprises a shape memory alloy (SMA). Non-limiting examples of shape memory alloys include nickel-titanium (i.e., nitinol).

Diagnostic catheters include, but are not limited to, angiography catheters, electrophysiology catheters, intravenous ultrasound catheters and the like.

Catheter angiography can be performed using such techniques as, for example, X-rays, computed tomography (CT) and magnetic resonance imaging (MRI). In catheter angiography, a catheter is inserted into a blood vessel (e.g., an artery) through a small incision in the skin. The catheter is guided to the area being examined, a contrast material is injected through the catheter and images are acquired using a small dose of ionizing radiation (e.g., X-rays). Contrast agents include, but are not limited to, iodinated low-osmolar contrast media (LOCM) and high-osmolar contrast media (HOCM). Low-osmolar contrast media include, but are not limited to, ioxaglate, iopamidol, iohexol, ioidixanol, iotrolan, ioxaglate, ioxilan, iopromide, ioversol and iomeprol. Non-limiting examples of high-osmolar contrast media include diatrizoate, metrizoate and iothalamate.

Catheter electrophysiology is an invasive heart catheterization that is designed to evaluate the electrical system of the heart. This test evaluates if there is a need for implantation of a pacemaker or defibrillator or to perform a catheter ablation which is a procedure that uses radiofrequency energy (similar to microwave heat) to destroy small areas of heart tissue that cause rapid or irregular heartbeats. In this procedure, a catheter is introduced into a blood vessel and placed under X-ray guidance into the heart. For example, catheter electrophysiology is used for evaluating patients who have concerning symptoms such as fainting, episodes of almost fainting, sensations of rapid heartbeats, or excessively slow heartbeats.

Ultrasound catheterization, or intravascular ultrasound (IVUS) is an imaging procedure using a catheter with a miniaturized ultrasound probe attached to the distal end. The proximal end of the catheter is attached to computerized ultrasound equipment which measure how sound waves reflect off blood vessels and converts these measurements into images. IVUS is used to determine, among others, the accumulation of plaque in an artery and the correct placement of a stent.

According to some embodiments, the diagnostic catheter ranges in diameter from about 0.1 French (Fr) to about 12 French (Fr).

The term “therapeutic catheter” includes, but is not limited to, a proximal endovascular thrombectomy catheter, a distal endovascular thrombectomy catheter, a self-expanding stent catheter, a retrievable thrombectomy stent catheter, an ablation catheter, a percutaneous transluminal angioplasty (PTCA) catheter, an embolization and the like.

PTCA is a minimally invasive procedure to open blocked coronary arteries, allowing blood to circulate unobstructed to the heart muscle. The procedure begins with the injection of local anesthesia into the groin area and putting a needle into the femoral artery. A guide wire is placed through the needle and the needle is removed. An introducer is then placed over the guide wire, after which the wire is removed. A different sized guide wire is then put in its place. Next, a long narrow tube called a diagnostic catheter is advanced through the introducer over the guide wire, into the blood vessel. This catheter is then guided to the aorta and the guide wire is removed. Once the catheter is placed in the opening (or ostium) of one the coronary arteries, a contrast dye is injected and an x-ray is taken. If a treatable blockage is noted, the first catheter is exchanged for a guiding catheter. Once the guiding catheter is in place, a guide wire is advanced across the blockage, then a balloon catheter is advanced to the blockage site. The balloon is inflated for a few seconds to compress the blockage against the artery wall. Then the balloon is deflated.

Catheter embolization is a minimally invasive treatment that occludes or blocks one or more blood vessels or vascular channels of malformations (abnormalities). In a catheter embolization procedure, medications or synthetic materials (embolic agents) are placed through a catheter into a blood vessel to prevent blood flow to the area. Using image-guidance, a catheter is inserted through the skin to the treatment site. A contrast material is then injected through the catheter and a series of x-rays are taken to locate the exact site of bleeding or abnormality. Next, a medication or an embolic agent is injected through the catheter. Additional x-rays are taken to ensure the loss of blood flow in the target vessel or malformation. Uses of catheter embolization include, but are not limited, control or prevention of abnormal bleeding, including bleeding that results from an injury, tumor or gastrointestinal tract lesions such as an ulcer or diverticular disease; controlling bleeding into the abdomen or pelvis caused by traumatic injuries; treatment of long menstrual periods or heavy menstrual bleeding that results from fibroid tumors of the uterus; to occlude or close off vessels that are supplying blood to a tumor, to eliminate an arteriovenous malformation (AVM) or arteriovenous fistula (AVF) (abnormal connection or connections between arteries and veins); and to treat aneurysms (a bulge or sac formed in a weak artery wall) by either blocking an artery supplying the aneurysm or closing the aneurysmal sac itself.

The various components of the described invention may be comprised of one or more materials. For example, according to some embodiments, the components can comprise one or more of a thermoplastic, a thermoset, a composite or a radiopaque filler.

Thermoplastics include, but are not limited to, nylon, polyethylene terephthalate (PET), urethane, polyethylene, polyvinyl chloride (PVC) and polyether ether ketone (PEEK).

Thermosets include, but are not limited to, silicone, polytetrafluoroethylene (PTFE) and polyimide.

Composites include, but are not limited to, liquid crystal polymers (LCP). LCPs are partially crystalline aromatic polyesters based on p-hydroxybenzoic acid and related monomers. LCPs are highly ordered structures when in the liquid phase, but the degree of order is less than that of a regular solid crystal. LCPs can be substituted for such materials as ceramics, metals, composites and other plastics due to their strength at extreme temperatures and resistance to chemicals, weathering, radiation and heat. Non-limiting examples of LCPs include wholly or partially aromatic polyesters or copolyesters such as XYDAR® (Amoco) or VECTRA® (Hoechst Celanese). Other commercial liquid crystal polymers include SUMIKOSUPER™ and EKONOL™ (Sumitomo Chemical), DuPont HX™ and DuPont ZENITE™ (E.I. DuPont de Nemours), RODRUN™ (Unitika) and GRANLAR™ (Grandmont).

Non-limiting examples of radiopaque fillers include barium sulfate, bismuth oxychloride, tantalum and the like.

According to some embodiments, the working lumen formed by the side-hole 310 and the first segment 320 further comprises a device separate from the endovascular device 300. According to some embodiments, the separate device is capable of being advanced into a blood vessel through the side-hole 310. According to some embodiments, the blood vessel is an artery or a vein. According to some embodiments, the separate device is an introducer. According to some embodiments, the introducer is rigid. According to some embodiments, the introducer is effective to straighten a catheter comprising a soft angled extension. According to some embodiments, the introducer and the straightened catheter comprising the soft angled extension are advanced through the working lumen formed by the side-hole 310 and the first segment 320; the introducer is removed from the working lumen formed by the side-hole 310 and the first segment 320; and the soft angled extension of the catheter pushes through the side-hole 310. According to some embodiments, the side-hole 310 directs the soft angled extension of the catheter into a blood vessel. According to some embodiments, the blood vessel is an artery or a vein.

According to some embodiments, diameter of the side-hole 310 is larger than diameter of the soft angled extension of the catheter. According to some embodiments, the side-hole 310 ranges in diameter from about 4 French (Fr) to about 12 French (Fr).

According to some embodiments, the side-hole 310 comprises an angled extension. According to some embodiments, the primary opening 160 comprises an angled extension. According to some embodiments, the angle of the angled extension is fixed. According to some embodiments, the angle of the angled extension ranges from about 0 degrees to about 359 degrees. According to some embodiments, the angle of the angled extension is adjustable. According to some embodiments, the angle of the angled extension is adjustable from about 0 degrees to about 359 degrees. According to some embodiments, the angled extension is adjusted after the endovascular device 300 is inserted into a blood vessel.

According to some embodiments, the catheter and/or angled extension may further include wires or cables within the wall that are capable of actively steering the device and creating bends in vivo.

According to some embodiments, the described invention can be used in an endovascular procedure in a subject suffering from an anatomical variation in a blood vessel. According to some embodiments, the described invention can be used in an endovascular procedure to treat acute stroke in a subject suffering from an anatomical variation in a blood vessel. According to some embodiments, the blood vessel comprises an anatomical variation comprising tortuosity. According to some embodiments, the blood vessel comprises an anatomical variation comprising an acute angulation. According to some embodiments, the acute angulation is an aortic arch variation. According to some embodiments, the aortic arch variation is a bovine arch variation. According to some embodiments, the acute angulation is a vertebral artery variation.

According to some embodiments, the support lumen 110 and/or the second segment 330 is advanced through the Subclavian artery into the arm, or alternatively, into the external carotid artery. According to some embodiments, the support lumen 110 and/or the second segment 330 provides support for a catheter, a wire or a combination thereof, advanced through the working lumen 120 and/or the working lumen formed by the side-hole 310 and the first segment 320 and into a blood vessel. According to some embodiments, the blood vessel is the left internal carotid artery. According to some embodiments, the blood vessel is the distal vertebral artery. According to some embodiments, the support lumen 110 and/or the second segment 330 prevents kickback of an advancing catheter, an advancing wire or a combination thereof.

According to some embodiments, there is a single side-hole. According to some embodiments, there are multiple side-holes. According to some embodiments, with multiple side-holes, all holes are along the same length of said catheter, at different circumferential locations. According to some embodiments, with multiple side-holes, all side-holes are staggered at different lengths along said catheter, either along the same or various circumferential locations. According to some embodiments, with multiple side-holes, some side-holes are staggered at different lengths along said catheter and some are along the same length. According to some embodiments, side-holes may be long partially overlapping lengths of said catheter, along varying circumferential locations.

Where a range of values is provided, it is understood that each intervening value, to the tenth of the unit of the lower limit unless the context dearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

1-55. (canceled)

56. A method of treating an intracranial aneurysm in a patient, wherein the method comprises:
providing an endovascular device, wherein the endovascular device includes:
a proximal segment;
a distal segment including a tapered outer diameter and a constant inner diameter; and
at least one side-hole located between the proximal segment and the distal segment;
introducing a guide catheter into a precerebral artery;
advancing an endovascular device through the guide catheter such that endovascular device spans the intracranial aneurysm;
positioning the at least one side-hole adjacent to a neck of the intracranial aneurysm;
confirming that the at least one side-hole is centered on the intracranial aneurysm via imaging;
positioning the distal segment in apposition with the precerebral artery to thereby anchor the endovascular device and reduce kickback and/or prolapse;
advancing a microcatheter into the intracranial aneurysm through the proximal segment and the at least one side-hole;
advancing an aneurysm treatment device through the microcatheter and into the intracranial aneurysm;
deploying the aneurysm treatment device;
removing the microcatheter;
removing the endovascular device; and
removing the guide catheter.

57. The method of claim 56, further comprising:
inflating at least one balloon on the endovascular device to further anchor the endovascular device within the precerebral artery.

58. The method of claim 57, wherein inflating the at least one balloon includes inflating the at least one balloon via a lumen substantially within a wall of the endovascular device utilized solely for inflation and deflation of the at least one balloon.

59. The method of claim 56, further comprising:
positioning an angled extension adjacent to the intracranial aneurysm, wherein the angled extension protrudes from the at least one side-hole.

60. The method of claim 59, further comprising:
activating a shape memory polymer to actively adjust the angled extension and direct one or more additional devices into the precerebral artery.

61. A method of treating an intracranial aneurysm in a patient, wherein the method comprises:
introducing a guide catheter into a precerebral artery via a vascular access site;
introducing a delivery device into the precerebral artery;
advancing an endovascular device through the guide catheter over the delivery device such that a side-hole of the endovascular device is positioned substantially adjacent to the intracranial aneurysm and such that an end-hole of the endovascular device is positioned distally of the intracranial aneurysm;
rotating the endovascular device such that the side-hole of the endovascular device is oriented towards the intracranial aneurysm;
removing the delivery device;
advancing a microcatheter through a first segment of the endovascular device and into the intracranial aneurysm through the side-hole, wherein the first segment extends proximally of the side-hole;
anchoring the endovascular device to inhibit kickback and/or prolapse;
advancing an aneurysm treatment device through the microcatheter and into the intracranial aneurysm, wherein the aneurysm treatment device is attached to a delivery system;
deploying the aneurysm treatment device;
performing radiographic confirmation of positioning of the aneurysm treatment device;
detaching the aneurysm treatment device from the delivery system;
removing the microcatheter;
removing the endovascular device;
removing the guide catheter; and
establishing hemostasis at the vascular access site.

62. The method of claim 61, wherein introducing the guide catheter into the precerebral artery includes advancing the guide catheter into a vertebral artery.

63. The method of claim 61, wherein introducing the guide catheter into the precerebral artery includes advancing the guide catheter into an internal carotid artery.

64. The method of claim 61, wherein introducing the delivery device into the precerebral artery includes introducing a delivery wire into the precerebral artery.

65. The method of claim 61, wherein introducing the delivery device into the precerebral artery includes introducing a delivery catheter into the precerebral artery.

66. The method of claim 61, wherein advancing the microcatheter through the first segment of the endovascular device includes advancing the microcatheter over a wire.

67. The method of claim 61, wherein anchoring the endovascular device includes positioning a second segment thereof in apposition with the precerebral artery, wherein the second segment extends distally from the side-hole.

68. The method of claim 61, further comprising:
performing radiographic confirmation of positioning of the aneurysm treatment device.

69. A method of treating an arteriovenous malformation (AVM) in a patient, wherein the method comprises:
advancing an endovascular device into an artery such that a side-hole of the endovascular device is positioned substantially adjacent to a vessel attached to the AVM, wherein the endovascular device includes:
a first segment extending proximally from the side-hole; and
a second segment extending distally from the side-hole;
positioning the endovascular device such that the side-hole is oriented towards the AVM;
advancing a microcatheter through a side-hole of the endovascular device and into the vessel attached to the AVM;
anchoring the endovascular device within the artery to inhibit kickback and/or prolapse;
advancing a treatment device through the microcatheter and into the AVM;
deploying the treatment device;
removing the microcatheter; and
removing the endovascular device.

70. The method of claim 69, wherein advancing the endovascular device into the artery includes advancing the endovascular device into a left middle cerebral artery.

71. The method of claim 69, wherein positioning the endovascular device includes rotating the endovascular device such that the side-hole is oriented towards a branch feeder of the AVM, and wherein advancing the microcatheter through the side-hole includes advancing the microcatheter into the branch feeder of the AVM.

72. The method of claim 69, wherein anchoring the endovascular device includes positioning the second segment in apposition with the artery to thereby reduce kickback and/or prolapse.

73. The method of claim 72, wherein advancing the endovascular device into the artery includes positioning the second segment distally of the AVM, wherein the second segment includes a tapered outer diameter and a constant inner diameter.
